SEQUENCE LISTING (8 sequences)

SEQ ID NO: 1 - protein, 186 aa, Homo sapiens
GMTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKC

SEQ ID NO: 2 - protein, 141 aa, Homo sapiens
SKTSNTIRVFLPNKQRTVVNVRNGMSLHDCLMKALKVRGLQPECCAVFRLLHEHKGKKARLDWNTDAASLIGEELQVDFLDHVPLTTHNFARKTFLKLAFCDICQKFLLNGFRCQTCGYKFHEHCSTKVPTMCVDWSNIRQ

SEQ ID NO: 3 - protein, 648 aa, Homo sapiens [sequence omitted]

SEQ ID NO: 4 - protein, 766 aa, Homo sapiens [sequence omitted]

SEQ ID NO: 5 - protein, 606 aa, Homo sapiens [sequence omitted]

SEQ ID NO: 6 - protein, 192 aa, Homo sapiens
MEHIQGAWKTISNGFGFKDAVFDGSSCISPTIVQQFGYQRRASDDGKLTDPSKTSNTIRVFLPNKQRTVVNVRNGMSLHDCLMKALKVRGLQPECCAVFRLLHEHKGKKARLDWNTDAASLIGEELQVDFLDHVPLTTHNFARKTFLKLAFCDICQKFLLNGFRCQTCGYKFHEHCSTKVPTMCVDWSNIRQ

SEQ ID NO: 7 - protein, 6 aa, Artificial Sequence
  Description of Artificial Sequence Synthetic 6xHis tag
HHHHHH

SEQ ID NO: 8 - protein, 20 aa, Artificial Sequence
  Description of Artificial Sequence Synthetic His tag
  SITE (1)..(20): This sequence may encompass 1-20 “His” residues
HHHHHHHHHHHHHHHHHHHH

(19) United States
(12) Patent Application Publication
(10) Pub. No.: US 2023/0258642 A1
(43) Pub. Date: Aug. 17, 2023

(54) COMPOSITIONS AND METHODS FOR IDENTIFICATION OF MODULATORS OF RAS PROTEIN COMPLEXES

(71) Applicant: QUANTA THERAPEUTICS, INC., San Francisco, CA (US)

(72) Inventors: Elizabeth Vo, San Francisco, CA (US); Matthew Holderfield, Oakland, CA (US); Joshua Salafsky, San Francisco, CA (US)

(21) Appl. No.: 17/808,450

(22) Filed: Jun. 23, 2022

Related U.S. Application Data
(63) Continuation of application No. PCT/US2021/012356, filed on Jan. 6, 2021.
(60) Provisional application No. 62/957,786, filed on Jan. 6, 2020.
(60) Provisional application No. 62/984,761, filed on Mar. 3, 2020.
(60) Provisional application No. 63/068,120, filed on Aug. 20, 2020.

(51) Int. Cl. G01N 33/574; G01N 33/542

(52) U.S. Cl. CPC G01N 33/5748; G01N 33/542; G01N 2333/82; G01N 2333/912; G01N 2333/91188

(57) ABSTRACT

Provided herein are compositions and methods for detecting conformational changes in a protein using surface-selective techniques in conjunction with a cell-free support interface. The protein generally comprises a lipid tail. The compositions and methods are useful in identifying agents that modify the conformation and activity of such proteins and protein complexes.

### CROSS-REFERENCE

This application is a Continuation of International Patent Application No. PCT/US2021/012356, filed on Jan. 6, 2021, which claims benefit of U.S. Provisional Patent Application No. 62/957,786 filed on Jan. 6, 2020, U.S. Provisional Patent Application No. 62/984,761 filed on Mar. 3, 2020 and U.S. Provisional Patent Application No. 63/068,120 filed on Aug. 20, 2020, each of which is incorporated herein by reference in its entirety.

### SEQUENCE LISTING

The instant application contains a Sequence Listing which has been submitted electronically in XML format and is hereby incorporated by reference in its entirety. Said XML copy, created on Apr. 18, 2023, is named 55172-701.301SL.xml and is 23,034 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Many different types of cancer cells manipulate the GTPase and related signaling pathways for growth advantages partly due to the crosstalk with other fundamental pathways. Despite the major role played by mutant forms of these proteins in human cancer, many proteins are widely considered an undruggable target due to the picomolar affinity for GTP, GTP's role in several significant cellular processes unrelated to a particular protein target of interest, as well as the critical role of protein-protein interactions between the protein target and potential accessory proteins in a signaling cascade. What is needed, therefore, are techniques to identify compounds capable of specifically modulating the behavior of proteins and protein complexes in cancer cells to decrease the aberrant signaling of downstream effector molecules. Such techniques are provided herein by the disclosure of methods for identifying agents capable of modulating the structure of proteins and protein complexes into alternate conformational states.

The Ras pathway is one such example. Ras proteins are small GTPases that regulate cell differentiation, growth, and proliferation. Mutant KRAS is one of the most common driver oncogenes. KRAS mutations often occur earlier in tumor genesis and a variety of KRAS mutations are implicated in many cancers. Mutant KRAS is often required for tumor cell survival. Despite this critical role in oncogenesis, Ras is considered “undruggable” using traditional active-site drug discovery approaches due to the picomolar affinity of GTP, Ras's natural substrate.

Traditional drug discovery approaches aim to inhibit Ras activity at the evolutionarily conserved active site by competing with the natural substrate, which raises additional challenges (e.g., reduced selectivity). Furthermore, traditional biophysical discovery methods are slow and low-throughput. The methods are not well suited to discovery of chemical modulators that bind weakly to novel pockets or that can work across a spectrum of Ras mutants. Currently, therefore, there is a need for effective discovery platforms that screen for non-covalent chemical entity leads for targets like KRAS.

### SUMMARY

In certain aspects the disclosure provides a polypeptide composition comprising a first polypeptide covalently bound to a lipid tail; a second polypeptide non-covalently contacting the first polypeptide at an interface, wherein at least one of the first polypeptide and second polypeptide is covalently bound to an SHG-active label; and a membrane-like substrate, wherein the lipid tail of said first polypeptide non-covalently contacts the membrane-like substrate to secure the first polypeptide and second polypeptide to the membrane-like substrate. In certain embodiments, the first polypeptide comprises a GTPase or a fragment thereof. In certain embodiments, the second polypeptide comprises a PI3K protein or a fragment thereof or a RAF protein or fragment thereof.

In certain aspects the disclosure proves a polypeptide composition comprising a first polypeptide covalently bound to a lipid tail and the first polypeptide is further covalently bound to an SHG-active label; and a membrane-like substrate, wherein the lipid tail of said first polypeptide non-covalently contacts the membrane-like substrate to secure the first polypeptide to the membrane-like substrate.

In certain aspects the disclosure provides a method for identifying an agent that causes a conformational change in a polypeptide complex wherein the method provides the steps of: (a) contacting a polypeptide complex as disclosed herein with a test agent; (b) measuring an SHG signal from the SHG-active label; and (c) comparing the SHG signal to an SHG signal measured in an absence of the test agent, wherein a change in an amount of SHG signal in the presence of the test agent indicates that the test agent is an agent that causes a conformational change in the polypeptide complex. In certain embodiments, the methods described herein provide a method of treating cancer by providing administering a test agent to a subject in need thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-C show characterization of RAF1^{RBDCRD}-SHG2 conjugate. FIG. 1A shows the cysteine amino acids labeled by SHG probe on an exemplary RAF1. Figure discloses SEQ ID NO: 6. FIG. 1B shows a mass spectrometry peptide analysis indicating that a majority (>80%) of the RAF1^{RBDCRD}-SHG2 protein conjugates carry one dye molecule per protein molecule. FIG. 1C shows a schematic of the SHG complex assay showing KRAS-FMe: RAF1^{RBDCRD} anchored to a phosphatidylserine (PS) enriched lipid bilayer.

FIG. 2A-B show further functional characterization of RAF1^{RBDCRD}-SHG2 conjugate. FIG. 2A shows the SHG signal change upon addition of SH-active RAF1 conjugates in the presence of KRAS^{G12D}-FMe bound to a PS bilayer. FIG. 2B shows the SHG signal change upon addition of SH-active RAF1 conjugates on PS bilayer alone.

FIG. 3A-B show SHG detection of RAF1 binding to KRAS^{G12D}-FMe on a PS bilayer. FIG. 3A shows SHG intensity when monitored 30-minutes post the addition of either equimolar SH-active RAF1^{RBDCRD} or buffer to KRAS^{G12D}-FMe tethered overnight to a PS bilayer. FIG. 3B shows the baseline SHG intensity from a pre-formed KRAS^{G12D}-FMe+SH-active RAF1 complex captured on a PS bilayer. A PS bilayer was incubated overnight with each individual protein component or the complex as described, and baseline SHG was measured after washing off the untethered protein from bilayer with assay buffer.

FIG. 4 shows the assay buffer optimization for screening of the KRAS^{G12D}-FMe+SH-active RAF1 complex for screening. The assay buffer was optimized with addition of TCEP and GppNHp to prevent RAF1RBDCRD-SHG2 dissociation over time.

FIG. 5A shows that addition of EDTA and GDP causes dissociation of the complex which is measured as a robust decrease in the SHG signal in the first 30 minutes and maintained over 120 minutes. FIG. 5B shows that addition of EDTA and GDP cause dissociation of the complex which is measured as a robust decrease of over 80% in the SHG signal.

FIG. 6 shows the spread of SHG signal response from unknown chemical entities in a library as compared to control conditions (DMSO and EDTA+GDP) allowing identification of either complex disruptors or conformational modulators.

FIG. 7A-B shows the development of a counter screen assay to flag false positives using Annexin V. FIG. 7A shows the SHG signal from addition of chemical modulators to a KRAS-RAF1^{RBDCBD} complex compared to an Annexin V tethered to a lipid bilayer. FIG. 7B shows the change in SHG signal upon addition of K201 to Annexin V complex tethered to a bilayer.

FIG. 8 shows a schematic of different mechanistic profiles of chemical agents that can affect KRAS^{G12D}-FMe+SH-active RAF1 complex. FIG. 8 depicts Mechanism 1 showing a schematic of a putative small molecule agent that produces a change in SHG signal by causing dissociation of the KRAS^{G12D}-FMe+SH-active RAF1 complex. FIG. 8 depicts Mechanism 2 showing a schematic of a putative small molecule agent that produces a change in SHG signal by a conformational change in the KRAS^{G12D}-FMe+SH-active RAF1 complex.

FIG. 9A-C shows a schematic of different mechanistic profiles of chemical agents that can affect KRAS^{G12D}-FMe+SH-active RAF1 complex. FIG. 9A shows a schematic of the binding kinetics of a putative small molecule agent that produces a change in SHG signal by causing dissociation of the KRAS^{G12D}-FMe+SH-active RAF1 complex. FIG. 9B shows a schematic of the binding kinetics of a putative small molecule agent that produces a change in SHG signal by causing a conformational change in the KRAS^{G12D}-FMe+SH-active RAF1 complex. FIG. 9C shows two examples of potentially spurious small molecule SHG binding kinetics. The top graph shows a SHG signal change that does not stabilize over 60 minutes, which is generally indicative of a non-specific binder or aggregator. The bottom graph shows a small molecule that failed to produce a significant SHG response at all concentrations and moreover showed high variability. Based on these results, these molecules and others with similar SHG responses were not pursued further for characterization.

FIG. 10 depicts the confirmation of the mechanism of action of a putative small molecule that produces a change in SHG signal by causing dissociation of the KRAS^{G12D}-FMe+SH-active RAF1 complex using alternative orthogonal structural methods.

FIG. 11 depicts the confirmation of the mechanism of action of a putative small molecule that produces a change in SHG signal by causing a conformational change in the KRAS^{G12D}-FMe+SH-active RAF1 complex using alternative orthogonal structural methods.

### DETAILED DESCRIPTION

### I. Overview

The embodiments provided herein include, inter alia, compositions and methods for identifying and detecting modulators of conformational states of proteins and protein complexes (for example, Ras/Raf protein complexes) through the use of second harmonic generation technology and in conjunction with a system that includes supported cell-free bilayers. In contrast to the traditional active-site based approach, the compositions and methods herein provide a structure-based allosteric approach for identifying and detecting modulators of conformational states of membrane-associated proteins or protein complexes (for example, Ras/Raf protein complex), thereby unlocking the druggability of these targets. For example, allosteric modulators can activate or inhibit target activity and can bind to sites other than the natural substrate binding active site. Potential binding sites can be unique or less conserved sites, thereby allowing for better selectivity of downstream effects.

KRAS mutated cancer cell lines can also be also RAF1 dependent, making RAF1 a viable target for KRAS driven cancers. Provided here are compositions and methods to identify agents using second harmonic generation technology that alter RAF/RAS activity. Such agents can alter RAF/RAS binding, induce conformational changes in an oncogenic KRAS-RAF1 complex, or alter downstream signaling. In embodiments, the KRAS-RAF1 comprises active oncogenic KRAS^{G12D}-RAF1 complex immobilized by the C-terminal farnesyl tail of KRAS^{G12D} to a cell-free support. In embodiments, the methods provided herein allow screening of novel agents that induce conformational changes against a protein complex target comprising KRAS protein complexed with a RAF1 effector attached to a second harmonic label (SH-activated RAF1) and immobilized to a lipid surface mimicking its physiological conformational state. In embodiments, the SH-activated RAF1 construct includes both the Ras binding domain (RBD) and the cysteine rich domain (CRD), providing an opportunity to identify novel pockets at the interface of Ras and RAF1 in the presence of a lipid surface.

The methods of the present disclosure provide techniques permitting the identification of agents that can modulate the behavior of a membrane-bound protein complex (e.g., a Ras/Raf complex). The methods of the present disclosure provide techniques permitting the identification of agents that can allosterically affect the conformation of a membrane-bound protein complex (e.g., a Ras/Raf complex). In embodiments, the agent can allosterically affect the binding affinity of proteins constituting a membrane-bound protein complex (e.g., a Ras/Raf complex). The compositions and methods herein are useful for assessing conformational changes in a membrane-based platform where physiologically relevant structure-function relationships are preserved.

Additionally, provided herein are methods for using SHG-based techniques in conjunction with supported lipid bilayer systems to detect extremely subtle conformational changes, on the order of angstroms or sub-angstroms, in protein complexes bound to a surface which supports a lipid bilayer using SHG within the three-dimensional structure of proteins in general. Furthermore, the compositions and methods herein provide for high-throughput, sensitive, real-time direct readout of conformational changes in a protein complex. Therefore, the present disclosure describes methods which rapidly permit the determination of alterations in the three-dimensional structures of protein complexes without the time and labor-intensive processes associated with NMR and X-ray crystallography.

### II. Polypeptide Compositions

A. First Polypeptides Bound to a Surface

Provided herein are compositions useful for detecting conformational changes in a membrane-tethered polypeptide composition using a surface-selective non-linear optical technique. In some aspects, the polypeptide composition comprises a first polypeptide covalently bound to a lipid tail, the first polypeptide is further covalently bound to an SHG-active label; and a membrane-like substrate, wherein the lipid tail of said first polypeptide non-covalently contacts the membrane-like substrate to secure the first polypeptide to the membrane-like substrate.

In some cases, the first polypeptide of the polypeptide composition comprises a GTPase. In some cases, the GTPase is selected from the Ras superfamily or proteins or a fragment any one thereof. In some cases, the GTPase is selected from Ras, Rho, Rab, Rap, Arf, Ran, Rheb, RGK, Rit, and Miro, or a fragment of any one thereof. In some cases, the GTPase or fragment thereof has an inactive kinase domain or lacks a kinase domain.

In some cases, the polypeptide composition comprises a first polypeptide that is bound to a lipid tail. In some cases, the polypeptide composition comprises a first polypeptide covalently bound to a lipid tail. In cases, the lipid tail of the first polypeptide is covalently bound to a cysteine or a lysine of the first polypeptide. In some cases, the lipid tail comprises a prenyl group, a fatty acid group, or a glycosylphosphatidylinositol group. In some cases, the lipid tail of the polypeptide composition comprises a farnesyl group.

In some cases, the polypeptide composition comprises a first polypeptide that is bound to a SHG-active label. In some aspects, the SHG-active label is covalently bound to a cysteine or a lysine on the first polypeptide. In some cases, the SHG-active label is covalently bound to a cysteine or a lysine on the first polypeptide. In some cases, the SHG label comprises a SH-active dye. In some cases, the SH-active dye is selected from a maleimide label, PyMPO-NHS, an oxazole label, Badan, and Acrylodan. In some cases, the first polypeptide is covalently bound to between 1 and 8 SHG-active labels. In some cases, the polypeptide composition comprises a first polypeptide that is bound to both a SHG-active label and a lipid tail. In some cases, the polypeptide composition comprises a first polypeptide that is covalently bound to both a SHG-active label and a lipid tail. In some cases, a test agent contacts the first polypeptide. In some cases, the test agent is a small molecule ligand. In some cases, the test agent is a molecule with a molecular weight of less than 1000 AMU. In some cases, the polypeptide composition further comprises a small molecule ligand non-covalently bound to the first polypeptide. In some cases, the agent is bound to the first polypeptide at an interface. In some embodiments, the interface is a site on the first polypeptide that is capable of interacting with a second polypeptide. In some cases, the small molecule ligand induces a conformational change in the first polypeptide.

B. Polypeptide Complexes Bound to a Surface

Provided herein are compositions useful for detecting interactions between at least two components in a membrane-tethered polypeptide composition using a surface-selective non-linear optical technique. In some cases, the polypeptide composition comprises at least two polypeptides, such as a first polypeptide and a second polypeptide. In some embodiments, the first polypeptide and second polypeptide form a polypeptide complex. In some embodiments, the first polypeptide of the complex is as described above.

In some cases, the polypeptide composition comprises a protein complex comprising at least two proteins. In some cases, at least one of the proteins in the protein complex is tethered to a membrane. In some cases, at least one of the proteins in the protein complex is a naturally-occurring membrane bound protein. In some cases, at least one protein of the proteins in the membrane-tethered protein complex can be a protein of the Ras superfamily or a fragment thereof. In some cases, at least one protein of the proteins in the membrane-tethered protein complex can be a Raf kinase or a fragment thereof. Specific embodiments describing membrane-bound complexes comprising Ras or Raf proteins are described below.

In some cases, the polypeptide composition comprises a first polypeptide and a second polypeptide. In some cases, the polypeptide composition comprises a first polypeptide and a second polypeptide that is bound to a membrane-like substrate. In some cases, at least one of the polypeptides in the polypeptide complex is a naturally-occurring membrane bound polypeptide. In some cases, at least one polypeptide of the polypeptides in the membrane-tethered polypeptide complex can be a polypeptide of the Ras superfamily or a fragment thereof. In some cases, at least one polypeptide of the polypeptides in the membrane-tethered polypeptide complex can be a PI3K kinase or a fragment thereof or a RAF kinase or a fragment thereof. Specific embodiments describing polypeptide compositions bound to a membrane-like substrate are provided below.

In some aspects, the present disclosure provides a polypeptide composition comprising: a first polypeptide covalently bound to a lipid tail;

a second polypeptide non-covalently contacting the first polypeptide at an interface, wherein at least one of the first polypeptide and second polypeptide is covalently bound to an SHG-active label; and

a membrane-like substrate, wherein the lipid tail of said first polypeptide non-covalently contacts the membrane-like substrate to secure the first polypeptide and second polypeptide to the membrane-like substrate.

In some cases, the first polypeptide of the polypeptide composition comprises a GTPase. In some cases, the GTPase is selected from the Ras superfamily or proteins or a fragment any one thereof. In some cases, the GTPase is selected from Ras, Rho, Rab, Rap, Arf, Ran, Rheb, RGK, Rit, and Miro, or a fragment of any one thereof. In some cases, the GTPase or fragment thereof has an inactive kinase domain or lacks a kinase domain.

In some cases, the second polypeptide of the polypeptide composition comprises a PI3K protein or a fragment thereof or a RAF protein or fragment thereof. In some cases, the second polypeptide is a PI3K protein or fragment thereof. In some cases, the second polypeptide is selected from PIK3CA, PIK3CB, PIK3CG, PIK3CD, PIK3R1, PIK3R2, PIK3R3, PIK3R4, PIK3R5, PIK3R6, PIK3C2A, PIK3C2B, PIK3C2G, and PIK3C3, or a fragment of any one thereof. In some cases, the PI3K protein or fragment thereof of the second polypeptide has an inactive kinase domain or lacks a kinase domain. In some cases, the second polypeptide is a RAF protein or fragment thereof. In some cases, the second polypeptide is selected from A-RAF, B-RAF, and C-RAF, or a fragment of any one thereof. In some cases, the RAF protein or fragment thereof has an inactive kinase domain or lacks a kinase domain.

C. Lipid Tails

In some cases, the polypeptide composition comprises a first polypeptide that is bound to a lipid tail. In some cases, the polypeptide composition comprises a first polypeptide covalently bound to a lipid tail. In some cases, the first polypeptide is synthetically modified to provide a lipid tail binding domain. In some cases, the first polypeptide is synthetically modified to provide a lipid tail. In some cases, the synthetic modification is a post-translational modification that can occur at the N-terminus of the first polypeptide. In some cases, the lipid tail is added by lipidation. In some cases, the lipid tail is added by acylation, isoprenylation, N-myristoylation, S-palmitoylation, S-famesylation or S-geranylgeranylation. In some cases, the first polypeptide can be modified at the C-terminus by proteolysis or carboxyl methylation. In some cases, the first polypeptide can be modified at the C-terminus by conditional modifications selected from phosphorylation, peptidyl-prolyl isomerisation, monoubiquitylation, diubiquitylation, nitrosylation, ADP ribosylation and glucosylation. In some cases, the lipid tail of the first polypeptide is covalently bound to a cysteine or a lysine of the first polypeptide. In some cases, the lipid tail of the first polypeptide is covalently bound to a cysteine of the first polypeptide. In some cases, the lipid tail of the first polypeptide is covalently bound to a lysine of the first polypeptide. In some cases, the lipid tail comprises a prenyl group, a fatty acid group, or a glycosylphosphatidylinositol group. In some cases, the lipid tail of the polypeptide composition comprises a farnesyl group. In some cases, the farnesyl group of the first polypeptide is non-covalently bound to the membrane-like substrate.

D. Labels

Also provided herein are labels for use in labeling polypeptides. In some cases, at least one of the component polypeptides in a polypeptide complex in is labeled with a second harmonic-active moiety or label (a.k.a. an SH-active label or SHG-active label). In some cases, the SHG-active label is covalently bound to a cysteine or a lysine on the first polypeptide or second polypeptide. In some cases, the polypeptide composition comprises a first polypeptide that is bound to a SHG-active label. In some cases, the SHG-active label is covalently bound to the first polypeptide and the second polypeptide is not bound to a SHG-active label. In some cases, the polypeptide composition comprises a first polypeptide that is covalently bound to a SHG-active label. In some cases, the SHG-active label is covalently bound to a cysteine or a lysine on the first polypeptide. In some cases, the SHG label comprises a SH-active dye. In some cases, the SH-active dye is selected from a maleimide label, PyMPO-NHS, an oxazole label, Badan, and Acrylodan. In some cases, the polypeptide composition comprises a first polypeptide that is bound to both a SHG-active label and a lipid tail. In some cases, the polypeptide composition comprises a first polypeptide that is covalently bound to both a SHG-active label and a lipid tail.

In some cases, the first polypeptide can be SHG-active labeled by a dye through specific labeling or non-specific labeling. In some cases, the SHG-active label is a second harmonic-active moiety. The second harmonic-active moiety can be attached to a target protein through specific labeling, e.g., via a covalent bond or a hydrogen bond. For example, the second harmonic-active moiety (such as, a label) can be covalently or non-covalently attached to an amine group, a lysine group, or a sulfhydryl group in the primary amino acid sequence of the target protein to be detected. The sulfhydryl groups and/or amine groups can be native groups or engineered into the protein. In some cases, the second harmonic-active moiety (such as, a label) possesses an amine-reactive succinimidyl ester, a thiol-reactive maleimide, or an aldehyde- and/or ketone-reactive hydrazide and hydroxylamine. In some cases, the first polypeptide is covalently bound to between 1 and 8 SHG-active labels.

In some cases, polypeptide composition comprises a second polypeptide that is bound to a SHG-active label. In some cases, polypeptide composition comprises a second polypeptide that is covalently bound to a SHG-active label. the SHG-active label is covalently bound to the second polypeptide and the first polypeptide is not bound to a SHG-active label. In some cases, the SHG-active label is covalently bound to a cysteine or a lysine on the second polypeptide. In some cases, the SHG label comprises a SH-active dye. In some cases, the SH-active dye is selected from a maleimide label, PyMPO-NHS, an oxazole label, Badan, and Acrylodan.

In some cases, the second polypeptide can be SHG-active labeled by a dye through specific labeling or non-specific labeling. In some cases, the SHG-active label is a second harmonic-active moiety. The second harmonic-active moiety can be attached to a target protein through specific labeling, e.g., via a covalent bond or a hydrogen bond. For example, the second harmonic-active moiety (such as, a label) can be covalently or non-covalently attached to an amine group, a lysine group, or a sulfhydryl group in the primary amino acid sequence of the target protein to be detected. The sulfhydryl groups and/or amine groups can be native groups or engineered into the protein. In some cases, the second harmonic-active moiety (such as, a label) possesses an amine-reactive succinimidyl ester, a thiol-reactive maleimide, or an aldehyde- and/or ketone-reactive hydrazide and hydroxylamine. In some cases, the second polypeptide is covalently bound to between 1 and 8 SHG-active labels.

In some cases, the protein complex comprising a first polypeptide comprises at least two SH-active labels wherein the at least two SH-active labels may be attached to at least two or more amino acids in the first polypeptide. In some cases, at least 1, 2, 3, 4, 5, 6, 7, 8, 9, or 10 amino acids on the first polypeptide may be labeled with an SH-active label. In some cases, at least 1, 2, 3, 4, 5, 6, 7, 8, 9, or 10 SH-active labels may be attached to the first polypeptide. In embodiments, the SH-active labels are the same type of SH-active labels (e.g. dyes). In some embodiments, the SH-active labels are different types of SH-active labels. Examples of SH-active labels are provided elsewhere in this disclosure.

In some cases, the protein complex comprising a first polypeptide and a second polypeptide comprises at least two SH-active labels wherein the at least two SH-active labels are attached to at least two or more amino acids in the second polypeptide. In some cases, the two or more amino acids may be labeled with the same type of SH-active label. In some cases, the two or more amino acids may be labeled with two or more different types of SH-active labels. Examples of SH-active labels are provided elsewhere in this disclosure. In some cases, at least 1, 2, 3, 4, 5, 6, 7, 8, 9, or 10 amino acids on the second polypeptide may be labeled with an SH-active label. In some cases, at least 1, 2, 3, 4, 5, 6, 7, 8, 9, or 10 SH-active labels may be attached to the second polypeptide.

The degree of labeling can be determined, for example, by measuring absorbance of the dye and the protein and calculating a molar ratio using known extinction coefficients. In some cases, locations of the dye on a polypeptide can be determined by mass spectrometry.

In some cases, the polypeptide complex may comprise a Raf kinase or fragment thereof and a Ras polypeptide or fragment thereof. In some aspects, the polypeptide complex may consist of a Raf-1 kinase or fragment thereof and a KRAS polypeptide or fragment thereof.

Second harmonic-active moieties (such as, a label) can be bound, either covalently or non-covalently, to at least one of the constituent polypeptides of the polypeptide complex in order to render the resulting polypeptide complex susceptible to second harmonic generation and amenable to study at an interface using a surface-selective technique. The labeled polypeptide and polypeptide complex may then be studied by surface-selective techniques such as second harmonic generation or sum-frequency generation. In some aspects, a complex comprising a Ras protein or fragment thereof or a Raf protein or fragment thereof can be labeled with a second harmonic active label (SH-active label or SHG-active label), such as any of the labels described elsewhere in this disclosure. In embodiments, the Ras or Raf protein or fragments thereof in the protein complex are labeled with a second harmonic-active label on one or more of the protein's amino acid residues and attached to a surface or oriented at an interface, such as any of the surfaces or interfaces described herein, so that the SH-active label possesses a net orientation with respect to the interface.

In some cases, a component protein in the protein complex is labeled at two or more different amino acid locations with SH-active labels, such that a protein complex has at least one SH-active label attached to a protein in the complex. In some cases, the protein complex comprising a Raf kinase and a Ras protein may comprise at least one SH-active label wherein the at least one SH-active labels may be attached to the Raf kinase protein. In some cases, the Raf kinase protein or fragment has an amino acid sequence that comprises at least a portion of the sequence set forth in SEQ ID NO. 2. In some cases, the sequence can vary from the sequence set forth in SEQ ID NO: 2. In some cases, the at least one SH-active label may be attached to amino acids corresponding to cysteine 95 or cysteine 96 in SEQ ID NO: 2. In some cases, the at least one SH-active labels may be attached to amino acids corresponding to cysteine 133 as set forth in SEQ ID NO. 2.

In some cases, the at least one SH-active label may be attached to an amino acid in the Ras-binding domain of the Raf kinase protein. In some cases, the at least one SH-active labels may be attached to amino acids corresponding to cysteine 44 or cysteine 45 in SEQ ID NO. 3. In some cases, the at least one SH-active label may be attached to an amino acid in the cysteine-rich domain of the Raf kinase protein. In some cases, the at least one SH-active labels may be attached to amino acids corresponding to cysteine 184 in SEQ ID NO. 3.

In some cases, the protein complex comprising a Raf kinase and a Ras protein may comprise at least one SH-active label wherein the at least one SH-active labels may be attached to the Ras protein. In some cases, the Ras protein has a sequence as set forth in SEQ ID NO. 1. In some cases, the Raf kinase protein or fragment has an amino acid sequence that comprises at least a portion of the sequence set forth in SEQ ID NO. 3. In some cases, the sequence can vary from the sequence set forth in SEQ ID NO: 1. In some cases, the at least one SH-active label may be attached to an amino acid in the G domain (corresponding to amino acids 1-165) of the Ras protein (as set forth in SEQ ID NO. 1) as described above. In some cases, the at least one SH-active label may be attached to an amino acid in the C domain (corresponding to amino acids 165-189 in SEQ ID NO. 1) of the Ras protein as described above. In some cases, the at least one SH-active label may be attached to an amino acid in the switch I domain (corresponding to amino acids 32-38 in SEQ ID NO. 1) of the Ras protein as described above. In some cases, the at least one SH-active label may be attached to an amino acid in the switch II domain (corresponding to amino acids 59-67 in SEQ ID NO. 1) of the Ras protein as described above.

In some cases, the protein complex comprising a Raf kinase and a Ras protein or fragment thereof may comprise at least two SH-active labels wherein the at least two SH-active labels may be attached to at least two or more amino acids in the Raf kinase protein or fragment thereof. In some cases, at least 1, 2, 3, 4, 5, 6, 7, 8, 9, or 10 amino acids on the Raf kinase protein or fragment thereof may be labeled with an SH-active label. In some cases, at least 1, 2, 3, 4, 5, 6, 7, 8, 9, or 10 SH-active labels may be attached to the Raf kinase or fragment thereof. In embodiments, the SH-active labels are the same type of SH-active labels (e.g. dyes). In some embodiments, the SH-active labels are different types of SH-active labels. Examples of SH-active labels are provided elsewhere in this disclosure.

In some cases, the protein complex comprising a Raf kinase and a Ras protein may comprise at least two SH-active labels wherein the at least two SH-active labels may be attached to at least two or more amino acids in the Ras protein. In some cases, the two or more amino acids may be labeled with the same type of SH-active label. In some cases, the two or more amino acids may be labeled with two or more different types of SH-active labels. Examples of SH-active labels are provided elsewhere in this disclosure. In some cases, at least 1, 2, 3, 4, 5, 6, 7, 8, 9, or 10 amino acids on the Ras protein in the protein complex may be labeled with an SH-active label. In some cases, at least 1, 2, 3, 4, 5, 6, 7, 8, 9, or 10 SH-active labels may be attached to the Ras protein in the protein complex.

In some other cases, a protein in the protein complex can be labeled with two or more different SH-active labels, such that the protein complex carries at least two or more different SH-active labels at two or more different amino acid locations. For example, in some cases, in a protein complex comprising Raf-1 and at least one other protein, the SH-active labels can be attached to at least two different protein domains in the Raf kinase protein. In some cases, the SH-active labels in the Raf kinase are attached to amino acids that form an interaction interface with another constituent protein in the protein complex. In some cases, the protein may be an effector protein that binds to Raf kinase. Examples of such effector proteins are provided elsewhere in this disclosure. In some cases, the SH-active labels in the Raf kinase are attached to amino acids that form an interaction interface with a Ras protein in the protein complex.

In some cases, a protein in the protein complex can be labeled at two or more different amino acids locations with two or more different SH-active labels. For example, in some cases, the SH-active labels can be attached to at least two different amino acid locations in the Ras protein. In some cases, the SH-active labels in the Ras protein are attached to amino acids that form an interaction interface with another constituent protein in the protein complex. In some cases, the constituent protein may be an effector protein that binds to Ras protein. Examples of such effector proteins are provided elsewhere in this disclosure. In some cases, the SH-active labels in the Ras protein are attached to amino acids that form an interaction interface with a Raf-1 protein in the protein complex.

In some cases, two or more different constituent proteins in the protein complex can each be labeled with at least one SH-active label. In some cases, the at least two constituent proteins in the protein complex can be labeled with two or more different SH-active labels, such that the protein complex carries at two or more different SH-active labels at two or more different amino acid locations. For example, in some cases, the protein complex comprising a Raf kinase and a Ras protein may comprise at least two SH-active labels wherein at least one SH label is attached to the Ras protein and at least one SH-active label is attached to the Raf kinase. In some cases, the protein complex comprising a Raf kinase and a Ras protein may comprise at least 1, 2, 3, 4, 5, 6, 7, 8, 9, or 10 SH-active labels wherein the at least 1, 2, 3, 4, 5, 6, 7, 8, 9, or 10 SH-active labels are attached to the Ras protein and at least one SH-active label is attached to the Raf kinase.

In some cases, the protein complex may comprise at least one more protein in addition to Raf kinase or fragment thereof and Ras protein or fragment thereof. In some cases, the protein complex comprising Raf kinase or fragment thereof and Ras protein or fragment thereof may comprise at least 1, 2, 3, 4, 5, 6, 7, 8, 9, or 10 proteins. In some cases, the at least 1, 2, 3, 4, 5, 6, 7, 8, 9 or 10 constituent proteins in the protein complex may each comprise at least one SH-active label. In some cases, a constituent protein may be attached to a plurality of SH-active labels at 1, 2, 3, 4, 5, 6, 7, 8, 9, or 10 different regions within the constituent protein.

The labeled amino acids can include, but are not limited to, cysteine residues, lysine residues, or amines. In other embodiments, the Ras protein or Raf protein or fragments thereof is labeled with an unnatural amino acid, such as, but not limited to Aladan. In some embodiments, a native amino acid residue in the Ras or Raf protein or fragments thereof is labeled with the second harmonic active label. In other embodiments, the labeled amino acid residue can be a mutated or substituted amino acid residue (such as a conservatively mutated or a conservatively substituted amino acid residue) engineered into the primary amino acid sequence of the Ras or Raf protein or fragments thereof. In other embodiments, the Ras or Raf protein or fragments thereof is attached to a surface (such as any of the surfaces or interfaces described herein) and labeled with an SH-active label in situ.

In alternate aspects of the disclosure, at least two distinguishable second harmonic-active moieties (such as, labels) can be used. The orientation of the attached two or more distinguishable labels would then be chosen to facilitate well defined directions of the emanating coherent nonlinear light beam. The two or more distinguishable labels can be used in assays where multiple fundamental light beams at one or more frequencies, incident with one or more polarization directions relative to the sample, are used, with the resulting emanation of at least two nonlinear light beams. In one example, the second harmonic-active moiety (such as, a label) comprises a plurality of individual second harmonic-active moieties (such as, labels) which each have a nonlinear susceptibility and are bound together in a fixed and determinate orientation with respect to each other so as to increase the overall nonlinear susceptibility of the second harmonic-active moiety (such as, a label).

Labels can be attached to different types of labeling sites on a protein in the protein complex (e.g., Ras or Raf proteins or fragments thereof). In some examples, a native amino acid residue in the protein is labeled with the second harmonic-active label. The labeling sites can be located on the surface of the protein or buried within the protein. Preferably, the labeling sites are located on the protein surface. Labels attached to unnatural amino acids allow labeling a residue buried within the protein.

Labels can be attached to any type of amino acid residues. In some aspects, the labeled amino acid residues on the constituent protein of the protein complex can include, but are not limited to, cysteine residues, lysine residues, or amines. For example, the labels can be attached to a pre-selected site such as a cysteine residue or a lysine residue. The labels can also be randomly attached to the amino acids throughout the protein (e.g., via an amino group).

In some aspects of the instantly disclosed methods, a native amino acid residue in the primary amino acid sequence of a protein (e.g., Ras or Raf proteins or fragments thereof) in the protein complex can be mutated or substituted with another amino acid that is capable of binding to a second harmonic-active dye. In other examples, the labeled amino acid residue can be a mutated or substituted amino acid residue (such as a conservatively mutated or a conservatively substituted amino acid residue) engineered into the primary amino acid sequence of the target protein. For example, one or more labels can be attached to native protein residues or mutant protein sites (e.g., a site incorporating an unnatural amino acid), or a combination thereof.

In other examples, the target protein in the protein complex (e.g., Ras or Raf proteins or fragments thereof) is labeled with an unnatural amino acid, such as, but not limited to Aladan or Dansylalanine. In another example, the unnatural amino acid is sum-frequency generation-active (SFG-active). In some examples, a UAA comprising a unique probe with tailored vibrational properties can be engineered into a target protein at a discrete site (such as an interfacial zone of interaction between the target protein and another protein) to identify site-specific conformational changes by SFG.

Labels can bind to different residues (e.g., cysteine or lysine residues) at different rates or different occupancy. For example, labels in solution can bind to an amine group at one site faster than an amine group at another site. The binding rates also depend on the labeling reaction conditions. For example, a change in pH can mean that one type of residue will preferentially bind a given label. Further, the ratio of labels (e.g., dye molecules) to the proteins to be labeled can affect the number of sites that are labeled. Controlling the number and/or location of labeled sites can be important, for example, for probing the specificity of binding interactions. For unnatural amino acids, the labeling procedure can be specifically tailored for labeling selected sites. Such sites can be selected strategically, such as, for example, in the vicinity of functionally relevant sites.

The exogenous moieties (such as, a label) can be pre-attached to the protein in the protein complex, and any unbound or unreacted labels separated from the labeled entities before a measurement is made. In one example, the second harmonic-active moiety (such as, a label) is attached to the protein (e.g., Ras or Raf proteins or fragments thereof) of the protein complex in vitro. In some examples, the constituent proteins in the protein complex are labeled with an SH-active moiety (such as, a label) in situ (i.e., after being attached to the surface). In other examples, the constituent proteins in the protein complex (e.g., Ras or Raf proteins or fragments thereof) are labeled with an SH-active moiety (such as, a label) before being attached to a supported lipid bilayer surface or a supported lipid analog bilayer surface.

In some aspects, the SH-active label is bound to particular amino acid residues on the protein (e.g., Ras or Raf proteins or fragments thereof) known to bind one or more intracellular or extracellular ligands. These can include, without limitation, another protein, a peptide, a nucleic acid (such as an inhibitory nucleic acid, for example, an antisense oligonucleotide or an siRNA), a phospholipid, a carbohydrate, or a co-factor (such as, but not limited to, a metal ion or a vitamin). In one example, an SH-active label is bound to amino acids in a protein (e.g., Ras or Raf proteins or fragments thereof) known to be located in an interfacial zone of interaction between the target protein and another protein (i.e., located at a site of protein-protein interaction). In some cases, the amino acids may be located in an interfacial zone of protein-protein interaction between the Ras and Raf proteins or fragments thereof in the protein complex.

In embodiments, a protein constituting the protein complex is bound with a natural or synthetic ligand at its active site. Alternatively, the protein constituting the protein complex is isolated from a cell or tissue with a natural ligand already bound to its active site. In some embodiments, the synthetic ligand that is known to bind to the constituent protein's active site is a drug, such as an inhibitor or a synthetic analogue of the constituent protein's natural ligand. For example, in some cases, the protein constituting the protein complex can be a Ras protein. In some cases, the ligand can be GTP, GDP or an analog thereof. In some cases, the ligand can be complexed with an SH-active moiety (e.g., a dye). In some cases, the protein complex can comprise a Ras protein bound to a Raf kinase.

In some examples, one or more labeling sites can be made more available for labeling for example by immobilizing the protein to be labeled on a lipid bilayer, thus exposing a portion of the protein that faces away from the lipid bilayer. Thus, labeling can be performed randomly and/or specifically with respect to preselected locations on the protein. For example, 1, 2, 3, 4, 5, 6, 7, 8, 9, 10, 20, 30, 40, 50, 75, 100 or more labels can be provided near or surrounding a preselected location (such as a functionally relevant site). Assaying differently labeled proteins can provide a map of the protein conformational changes upon binding to a binding partner. In some methods, the labeling sites can be selected so that the sites form a predetermined pattern or grid on or within the protein. The conformational changes detected at different labeling sites can indicate specificity of the binding of the binding partner.

1. Second Harmonic-Active Moieties (Such as, Labels)

a. Second Harmonic-Active Dyes

In some aspects, the second harmonic-active moiety (such as, a label) is a dye. Examples of dyes appropriate for use as second harmonic or sum frequency-active moieties (such as, a label) in the methods disclosed herein include, without limitation, maleimide labels (such as PyMPO-MALEIMIDE™ (1-(2-Maleimidylethyl)-4-(5-(4-Methoxyphenyl)Oxazol-2-yl)Pyridinium Methanesulfonate), which specifically labels proteins on cysteine residues), PyMPO-NHS (which specifically labels lysine residues), oxazole labels (such as PyMPO-succinimidyl ester which specifically labels amines), BADAN™ (6-Bromoacetyl-2-Dimethylaminonaphthalene), and ACRYLODAN™ (6-Acryloyl-2-Dimethylaminonaphthalene). In other examples, the labels can be coumarin-based dyes such as, but not limited to, ketocoumarin, and 3,3-carbonyl bis (7-diethylaminocoumarin). In other examples, the label can be PyMPO-SE™ (1-(3-(Succinimidyloxycarbonyl)Benzyl)-4-(5-(4-Methoxyphenyl)Oxazol-2-yl)Pyridinium Bromide).

The target protein can be labeled by a dye through specific labeling or non-specific labeling. The second harmonic-active moiety can be attached to a target protein through specific labeling, e.g., via a covalent bond or a hydrogen bond. For example, the second harmonic-active moiety (such as, a label) can be covalently or non-covalently attached to an amine group, a lysine group, or a sulfhydryl group in the primary amino acid sequence of the target protein to be detected. The sulfhydryl groups and/or amine groups can be native groups or engineered into the protein. In some examples, the second harmonic-active moiety (such as, a label) possesses an amine-reactive succinimidyl ester, a thiol-reactive maleimide, or an aldehyde- and/or ketone-reactive hydrazide and hydroxylamine. In some examples, the SFG or SHG-active dye label can be conjugated via “click chemistry” for coupling to azides. Details of click chemistry for use in conjugate formation are described in: “Synthesis and Functionalization of Biomolecules via Click Chemistry, C. Schilling et al, Chapter 15 pages 355-378 in “Click Chemistry for Biotechnology and Materials Science” J. Lahann (Ed), Wiley (2009).

b. Unnatural Amino Acids

In other aspects, the second harmonic-active moiety (such as, a label) may be an unnatural amino acid (UAA). In some cases, UAAs can be used for labeling proteins (e.g., Ras or Raf proteins) at both buried and exposed sites. In some cases, UAAs can possess hyperpolarizability for detecting proteins using a nonlinear technique such as second harmonic generation. In one example, the unnatural amino acid is sum-frequency generation-active (SFG-active). As used herein, “sum-frequency generation-active” refers to an SH-active label that possess a hyperpolarizability and is detectable by SFG. In other examples, the UAA is not hyperpolarizable, but possesses the appropriate chemical functional group or groups to permit it to bind to a second harmonic-active label dye, such as any of the dyes described above. In another example, a UAA can be used to attach the protein in a protein complex (e.g., Ras or Raf protein or fragments thereof) to a surface, such that a second harmonic-active moiety (such as, a label) possesses a net orientation with respect to the surface.

In other examples, the UAA can include a probe with tailored vibrational properties for engineering into discreet sites within a protein to identify site-specific conformational changes by SFG. In some examples, probe moieties for inclusion into UAAs desirably are small enough so that they do not perturb native protein structure and can include, but are not limited to, NO, CN, SCN or N3. In some examples, the probe moieties provide unique vibrational signatures in the spectral range of between about 1,900 and 2,300 cm-1, which is well separated from intrinsic protein vibrations.

Any hyperpolarizable UAA can be used as a second harmonic-active moiety (such as, a label) to measure conformational changes in the structure of a target protein (e.g., Ras or Raf proteins) upon binding a candidate agent in any of the methods described herein. In some examples, the UAA is Aladan (Cohen et al., 2002, Science, 296:1700; Abbyad et al., 2007, J. Phys. Chem., 111:8269, the disclosures of which are incorporated herein by reference in their entireties). In other examples, the UAA is Dansylalanine (Summerer et al., Proc. Nat. Acad. Sci. U.S.A., 2006, 103(26): 9785-9789).

Accordingly, in some aspects, structural changes in the conformation of a protein (e.g., Ras or Raf proteins or fragments thereof) in a target protein complex can be determined in real time and real space by measuring the tilt angle or absolute tilt angle of an unnatural amino acid label, or a series of such labels, engineered into the amino acid sequence in different mutants of the protein in the target protein complex. The probes can be incorporated at any site within the target protein or at its termini, or in any domain thereof. In some examples, the protein can include a second harmonic-active label that is chemically equipped to react covalently with a UAA. For example, if the UAA incorporated into a protein in a protein complex is Para-acetyl-phenylalanine (pAcF), the second harmonic-active dye would have appropriate chemistry on it for bonding covalently to pAcF. In another example, the incorporation of a SHAA in addition to a second UAA, the second UAA (which will in general not be second harmonic-active) allows chemically orthogonal covalent coupling of the protein in an oriented manner to a surface derivatized with appropriate chemistry for reaction with the second UAA. With a highly oriented target protein sample that is SH-active (using the two UAA's), both the baseline SHG signal and the contrast (change in signal with conformational change) can be larger in comparison to target proteins which do not utilize UAA's to produce SHG signals.

In some cases, UAAs can be used as probes for detection of changes in protein structural confirmation (e.g., Ras or Raf proteins) is that the detection can be carried out in vivo—that is, in live cells. For example, the methods described herein can be used to detect the conformational change exhibited by a target protein in live cells in response to binding of a candidate agent. By using an oriented protein population of target proteins relative to a surface, a highly precise map of structure or conformational change in real space and real time can be built using target proteins containing a UAA as part of its amino acid sequence. In embodiments, substitution of one or more amino acid residues with a UAA in a primary amino acid sequence does not result in substantial changes in the susceptibility of a target protein encoded by that amino acid sequence to undergo a conformational change upon binding to GDP or GTP or upon hydrolysis of GTP or upon binding to an unknown candidate agent capable of binding a target protein and stabilizing it into either an inactive or active conformation.

In other aspects, use of one or more UAA's in the amino acid sequence of a target protein of a protein complex (e.g., Ras or Raf proteins) in any of the methods disclosed herein enables the determination of the actual conformational change the target protein in the protein complex undergoes upon binding to a candidate allosteric modulator by determining the tilt angle of one or more labels at one or more sites within the target protein as a function of time. The three dimensional structure of the target protein in the protein complex can be determined by making one or more mutants of a component protein in the protein complex each containing a SHAA probe placed in a different location (i.e., the probe orientation relative to the surface in each mutant, and therefore the side-chain orientation, can be determined for the probe in each mutant and a model of the overall three dimensional protein structure can be built using this information). Information from steric hindrance methods, statistical methods, molecular dynamics, Ramachandran plots, or energy minimization methods known to those skilled in the art can be used to further aid in determining the structure given the measured probe tilt angles. A time-resolved measurement of the tilt angle of a probe produces a motion picture of a conformational change of the protein(s) in the protein complex as it occurs in real time. Because of SHG's (and SFG's) virtually instantaneous response and sensitivity, spatial orientation of a particular probe (e.g., tilt angle or absolute tilt angle relative to a surface) can be measured in real time at almost any time scale of interest. Further information related to the use of UAA's in SHG techniques can be found in U.S. Patent Application Publication No.: 2010/0068144, the disclosure of which is incorporated herein by reference in its entirety.

E. Support Interfaces

In some aspects, the target protein complex (e.g., comprising Ras and Raf proteins or fragments thereof) is bound to a solid surface or oriented with respect to an interface such that a second harmonic-active-label bound to the protein complex has a net orientation. In some examples, the interface can be made of silica, glass, silicon, polystyrene, nylon, plastic, a metal, semiconductor or insulator surface, or any surface to which biological components can adsorb or be attached. In different examples, the interface can be a vapor-liquid interface, a liquid-liquid interface, a liquid-solid, or a solid-solid interface. In one example, the vapor-liquid interface is an air-water interface.

In one example, the liquid-liquid interface is an oil-water interface. In different examples, the liquid-solid interface is a water-glass interface or a benzene-SiO2 interface, with the lipid bilayer supported by the glass or the SiO2, respectively. Other exemplary materials having properties making them suitable for lipid bilayer-compatible surfaces include various glasses, silicon oxides, including oxidized silicon (SiO2), MgF2, CaF2, mica, photoresist, and various polymer films, such as thin polyacrylamide or dextran films.

In other examples, the solid surface can be a glass surface, a plastic surface, a metal surface, a latex surface, a rubber surface, a ceramic surface, a polymeric surface, a polypropylene surface, a polyvinylidene difluoride surface, a polystyrene surface, or a polyethylene surface (such as a polyethylene glycol surface). The support on which the target proteins are immobilized may be composed from a wide range of material, such as, but not limited to, biological, nonbiological, organic, inorganic, or a combination of any of these, existing as particles, strands, precipitates, gels, sheets, tubing, spheres, containers, capillaries, pads, slices, films, plates, or slides. The surface may have any convenient shape, such as, but not limited to, a disc, square, sphere, or circle.

The surface can be preferably flat but may also take on a variety of alternative surface configurations. For example, the surface may contain raised or depressed regions on which a sample (such as a protein) is located. The surface preferably forms a rigid support on which the sample can be formed. The surface is also chosen to provide appropriate light-absorbing characteristics. For example, the surface may be, without limitation, a polymerized Langmuir Blodgett film, functionalized glass, Si, Ge, GaAs, GaP, SiO2 SiN4, modified silicon, or any one of a wide variety of gels or polymers such as (poly)tetrafhioroefhylene, (poly)vinylidenedifluoride, polyethylene glycol, polystyrene, polycarbonate, or combinations thereof. Other surface materials will be readily apparent to those of skill in the art. In one example the substrate is flat glass or silica.

In some aspects, the surface can be etched using well known techniques to provide for desired surface features. For example, by way of the formation of trenches, v-grooves, mesa structures, or the like, the target proteins (such as, synthesis regions of proteins) may be more closely placed within the focus point of impinging light. The surface may also be provided with reflective “mirror” structures for maximization of emission collected therefrom. As another example, the surface can be divided into regions to form wells.

If a solid surface is used (e.g., planar substrate, beads, etc.) it can also be derivatized via various chemical reactions to either reduce or enhance its net surface charge density to optimize the detection of target protein-candidate allosteric modulator interactions. In another aspect of the present disclosure, oligo-polyethylene glycol (PEG) molecules can be used for immobilizing an affinity-tagged target protein to a surface for SHG or SFG detection. In some examples, the PEG can be SAT(PEG4) (N-Succinimidyl S-acetyl (thiotetraethylene glycol). A pegylated interface suitable for detecting SHG signals can be prepared by coating a suitable surface, such as any of the surfaces described above, with an oligo PEG solution. In one example the surface can be glass. In another example, the surface can be amino-terminated silane derivatized glass.

In another embodiment, the protein comprises an affinity tag. Affinity tags are common in the art and may be, for example, a histidine tag (such as a His6 tag (SEQ ID NO: 7)), a maltose binding protein tag, an HA tag, a biotin tag, a thiol tag, or a GST tag. In some examples, the affinity tag is a histidine having any of 1, 2, 3, 4, 5, 6, 7, 8, 9, 10, 11, 12, 13, 14, 15, 16, 17, 18, 19, 20, or more histidine residues (SEQ ID NO: 8). In some embodiments, the supported lipid bilayer comprises Ni-NTA-bearing lipids. In one example, the oligo-PEG molecules are modified with an agent that will bind to the affinity tag expressed on the target protein. The agent can be nickel, in the case of a histidine tag, or it can be a sugar (such as maltose), an antibody, or any other molecule known in the art that is capable of binding to an affinity tag.

In some aspects, the interface can also include biological cell and liposome surfaces. The attachment or immobilization can occur through a variety of techniques well known in the art. For example, with proteins, the surface can be derivatized with aldehyde silanes for coupling to amines on surfaces of biomolecules (MacBeath and Schreiber, 2000-relevant portions of which are incorporated by reference herein). BSA-NHS (BSA-N-hydroxysuccinimide) surfaces can also be used by first attaching a molecular layer of BSA to the surface and then activating it with N, N′-disuccinimidyl carbonate. The activated lysine, aspartate or glutamate residues on the BSA react with surface amines on the proteins.

F. Membrane-Like Substrates

In some cases, the polypeptide composition comprises a membrane-like substrate.

In some embodiments, the membrane-like substrate of the polypeptide composition comprises a lipid bilayer surface. In some cases, the lipid bilayer comprises a polar region and a non-polar region. In some cases, the lipid bilayer comprises a hydrophilic region and a hydrophobic region. In some cases, the lipid bilayer may be naturally derived and purified. In some cases, the lipid bilayer may be synthetically produced. In some cases, the lipid bilayer may be modified to increase the hydrophobicity at the surface of the bilayer. In some cases, the lipid bilayer may be modified to decrease the hydrophobicity at the surface of the bilayer. In some cases, the lipid bilayer can be modified to increase the covalent interactions between the lipid bilayer and lipid tail of the first polypeptide. In some cases, the lipid bilayer of the polypeptide composition comprises at least one of diacyl glycerol, phosphatidylcholine (PC), phosphatidylethanolamine (PE), phosphatidylserine (PS), phosphatidic acid, phosphatidylinositol (PI), phosphatidylglycerol (PG), sphingomyelin, or derivatives thereof.

In some cases the membrane-like substrate of the polypeptide composition comprises a first surface selected from a glass surface, a polyethylene glycol surface, a plastic surface, a metal surface, a latex surface, a rubber surface, a ceramic surface, a polymeric surface, a polypropylene surface, a polyvinylidene difluoride surface, a polyethylene surface, a semiconductor surface, silicon nitride surface, and titanium dioxide surface; and a second surface selected from a lipid bilayer surface and a lipid analog bilayer surface, wherein the lipid tail of said first polypeptide non-covalently contacts the second surface and the second surface is structurally supported by the first surface. In some cases, the second surface may be modified to increase the non-covalent interactions between the second surface and first surface. In some cases, the first surface may be modified to increase the non-covalent interactions between the first surface and second surface.

In yet another example, the surface is a supported lipid bilayer surface or a lipid analog bilayer surface. In some cases, the lipid bilayer surface or a lipid analog bilayer surface is attached to a solid support as described above. In some cases, the lipid bilayer surface or a lipid analog bilayer surface is unattached to a solid support as described above. A protein complex can be used in conjunction with a supported lipid bilayer and SHG-based techniques to measure conformational dynamics in that protein complex. In some aspects of the methods disclosed herein, an SHG-labeled protein complex is bound to a supported lipid bilayer so that the SHG-labeled protein is oriented with respect to an interface such that a second harmonic-active-label bound to the protein has a net orientation. It is this net orientation that can change upon binding of an agent capable of inducing a conformational change in the structure of the SHG-labeled protein. Supported phospholipid bilayers are well known in the art and there are numerous techniques available for their fabrication, with or without associated membrane proteins (Salafsky et al., Biochemistry, 1996-relevant portions of which are incorporated by reference herein by reference, “Biomembranes”, Gennis, Springer-Verlag, Kalb et al., 1992, and Brian et al., 1984, relevant portions of which are incorporated herein by reference).

To generate a solid surface that is “lipid bilayer-compatible,” the surface can typically be cleaned and/or treated to remove surface impurities (dirt, oils, etc.). The cleaning procedure can be frequently selected such that it does not substantially damage the functionality of the bilayer barrier regions. For example, embodiments where the interface made of photoresist should not be cleaned using the traditional pirhana solution acid wash (3:1 H₂SO₄:H₂O2), since the acid can strip off the bilayer barrier regions.

In embodiments, the supported bilayers typically can be submerged in aqueous solution to prevent their destruction when they become exposed to air. The aqueous film and bulk liquid phase may be any suitable aqueous solution, such as a buffered saline solution (e.g., PBS). The bulk solution can be readily changed by, e.g., flow-through rinsing with a solution having a different composition.

The supported bilayer itself can be a self-assembling, two-dimensional fluid system, typically consisting of two opposed leaflets of vesicle-forming lipid molecules. However, it can be constructed from any suitable membrane-forming amphiphile. Most bilayer-forming lipids are long-chain carboxylic acids, such as glycerides, having the hydroxyl groups of the glycerol esterified with (i) fatty acid chain(s), and (ii) a charged or polar moiety, such as a phosphate-ester group. The vesicle-forming lipids are preferably ones having two hydrocarbon chains, typically acyl chains, and a polar head group. Long-chain carboxylic acids with a phosphate group, or phospholipids, are particularly well-suited for use with the present disclosure.

There are a variety of synthetic vesicle-forming lipids and naturally-occurring vesicle-forming lipids, including the phospholipids, such as phosphatidylcholine (PC), phosphatidylethanolamine (PE), phosphatidylserine (PS), phosphatidic acid, phosphatidylinositol (PI), phosphatidylglycerol (PG), and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids include glycolipids and sterols such as cholesterol.

In embodiments, lipids or mixtures of lipids for use in preparing supported lipid bilayers or supported lipid analog bilayers on any of the surfaces disclosed herein can comprise phosphocholine such as, but not limited to, DOPC. In some embodiments, the lipid mixture can comprise, without limitation, one or more of DDAB (N,N-distearyl-N,N-dimethylammonium bromide), DMRIE (N-(1,2-dimyristyloxyprop-3-yl)-N,N-dimethyl-N-hydroxyethyl ammonium bromide), DODAC (N,N-dioleyl-N,N-dimethylammonium chloride), DOGS (diheptadecylamidoglycyl spermidine) DOPE (1,2-sn-dioleoylphoshatidyethanolamine), DOSPA (N-(1-(2,3-dioleyloxyl)propyl)-N-(2-(sperminecarboxamido)ethyl)-N,N-dimethylammonium trifluoroacetate), DOTAP (N-(1-(2,3-dioleyloxyl)propyl)-N,N,N-trimethylammonium chloride), DOTMA N-(1-(2,3-dioleyloxyl)propyl)-N,N,N-trimethylammonium chloride). In a further embodiment, any of the lipids or lipid components of lipid mixtures can include an affinity tag for binding to proteins (such as, but not limited to, a nicklated lipid, for example, DOGS—Ni-NTA lipids).

Preferred diacyl-chain lipids for use in the present disclosure include diacyl glycerol, phosphatidyl ethanolamine (PE) and phosphatidylglycerol (PG). These lipids are preferred for use as the vesicle-forming lipid, the major liposome component, and for use in the derivatized lipid described below. All of these phospholipids and others are available from specialized suppliers of phospholipids (e.g., Avanti Polar Lipids, Inc., Alabaster, Ala.) as well as from general chemical suppliers, such as Sigma Chemical Co. (St. Louis, Mo.).

### III. Agents for Modulating Target Proteins

In some aspects, the agents for use in the methods described herein can be candidate modulators of a target protein complex. The agents for use in the methods described herein can be any of a small molecule chemical compound, an antibody, a non-antibody polypeptide, a carbohydrate, an inhibitory nucleic acid, or any combination thereof. In some examples, the agent is an antibody (such as a humanized antibody) or a fragment thereof. Alternatively, the agent may be a small molecule compound. In other examples, the agent can be a non-antibody polypeptide (such as an isolated non-antibody polypeptide). In some examples, agent is a peptide (for example, an isolated peptide).

In some cases, the polypeptide composition further comprises a test agent. In some cases, a test agent contacts the polypeptide composition at the interface. In some cases, the test agent is a small molecule ligand. In some cases, the test agent is a molecule with a molecular weight of less than 1000 AMU. In some cases, the polypeptide composition further comprises a small molecule ligand non-covalently bound at the interface. In some cases, the small molecule ligand induces a conformational change in the polypeptide composition.

In some aspects, the present disclosure provides a polypeptide composition comprising: a GTPase or fragment thereof that is covalently bound to a lipid tail;

a second polypeptide non-covalently contacting the GTPase or fragment thereof at an interface, wherein at least one of the GTPase or fragment thereof and second polypeptide is covalently bound to an SHG-active label; and

a membrane-like substrate, wherein the lipid tail of said GTPase or fragment thereof non-covalently contacts the membrane-like substrate to secure the GTPase or fragment thereof and second polypeptide to the membrane-like substrate.

In some aspects, the present disclosure provides a polypeptide composition comprising: a GTPase or fragment thereof that is covalently bound to a lipid tail;

a Raf protein or fragment thereof non-covalently contacting the GTPase or fragment thereof at an interface, wherein at least one of the GTPase or fragment thereof and Raf protein or fragment thereof are covalently bound to an SHG-active label; and

a membrane-like substrate, wherein the lipid tail of said GTPase or fragment thereof non-covalently contacts the membrane-like substrate to secure the GTPase or fragment thereof and Raf protein or fragment thereof to the membrane-like substrate.

In some aspects, the present disclosure provides a polypeptide composition comprising: a GTPase or fragment thereof that is covalently bound to a lipid tail;

a second polypeptide non-covalently contacting the GTPase or fragment thereof at an interface, wherein at least one of the GTPase or fragment thereof and Raf protein or fragment thereof are covalently bound to an SHG-active label; and

a lipid bilayer, wherein the lipid tail of said GTPase or fragment thereof non-covalently contacts the lipid bilayer to secure the GTPase or fragment thereof and Raf protein or fragment thereof to the lipid bilayer.

In some aspects, the present disclosure provides a polypeptide composition comprising: a GTPase or fragment thereof that is covalently bound to a farnesyl group;

Raf protein or fragment thereof is non-covalently contacting the GTPase or fragment thereof at an interface, wherein at least one of the GTPase or fragment thereof and Raf protein or fragment thereof are covalently bound to an SHG-active label; and

a lipid bilayer, wherein the farnesyl group of said GTPase or fragment thereof non-covalently contacts the lipid bilayer to secure the GTPase or fragment thereof and Raf protein or fragment thereof to the lipid bilayer.

In some aspects, the present disclosure provides a polypeptide composition comprising: a GTPase or fragment thereof that is covalently bound to a lipid tail;

a PI3K protein or fragment thereof non-covalently contacting the GTPase or fragment thereof at an interface, wherein at least one of the GTPase or fragment thereof and PI3K protein or fragment thereof are covalently bound to an SHG-active label; and

a membrane-like substrate, wherein the lipid tail of said GTPase or fragment thereof non-covalently contacts the membrane-like substrate to secure the GTPase or fragment thereof and PI3K protein or fragment thereof to the membrane-like substrate.

In some aspects, the present disclosure provides a polypeptide composition comprising: a GTPase or fragment thereof that is covalently bound to a lipid tail;

a PI3K protein or fragment thereof non-covalently contacting the GTPase or fragment thereof at an interface, wherein at least one of the GTPase or fragment thereof and PI3K protein or fragment thereof are covalently bound to an SHG-active label; and

a lipid bilayer, wherein the lipid tail of said GTPase or fragment thereof non-covalently contacts the lipid bilayer to secure the GTPase or fragment thereof and PI3K protein or fragment thereof to the lipid bilayer.

In some aspects, the present disclosure provides a polypeptide composition comprising: a GTPase or fragment thereof that is covalently bound to a farnesyl group;

a PI3K protein or fragment thereof non-covalently contacting the GTPase or fragment thereof at an interface, wherein at least one of the GTPase or fragment thereof and PI3K protein or fragment thereof are covalently bound to an SHG-active label; and

a lipid bilayer, wherein the farnesyl group of said GTPase or fragment thereof non-covalently contacts the lipid bilayer to secure the GTPase or fragment thereof and PI3K protein or fragment thereof to the lipid bilayer.

A. Non-Antibody Binding Polypeptides

In some aspects, the agents for use in the methods described herein are non-antibody binding polypeptides. Binding polypeptides are polypeptides that bind, preferably specifically, to a target protein such as any of the target proteins described herein. Binding polypeptides may be chemically synthesized using known polypeptide synthesis methodology or may be prepared and purified using recombinant technology. Binding polypeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such binding polypeptides that are capable of binding, preferably specifically, to a wild type or mutant target protein.

The binding polypeptides can be modified to enhance their inhibitory effect (including, for example, enhanced affinity, improved pharmacokinetic properties such as half-life, stability, and clearance rate, reduced toxicity, etc.). Such modifications include, for example, glycosylation, pegylation, substitution with non-naturally occurring but functionally equivalent amino acid, linking groups, etc.

Binding polypeptides may be identified without undue experimentation using well known techniques. In this regard, it is noted that techniques for screening polypeptide libraries for binding polypeptides that are capable of binding to a polypeptide target are well known in the art.

B. Small Molecules

In some aspects, the agents for use in the methods described herein are small molecule chemical compounds. Small molecules are preferably organic molecules other than binding polypeptides or antibodies as defined herein that bind, preferably specifically, to a wild type or mutant target protein.

Organic small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, iso-cyanates, sulfonyl chlorides, diazo compounds, acid chlorides, or the like.

In some aspects, the small molecule chemical compound is a component of a combinatorial chemical library. Combinatorial chemical libraries are a collection of multiple species of chemical compounds comprised of smaller subunits or monomers. Combinatorial libraries come in a variety of sizes, ranging from a few hundred to many hundreds of thousand different species of chemical compounds. There are also a variety of library types, including oligomeric and polymeric libraries comprised of compounds such as carbohydrates, oligonucleotides, and small organic molecules, etc.

Organic small molecules may be identified and chemically synthesized using known methodology (see, e.g., PCT Publication Nos. WO00/00823 and WO00/39585). Organic small molecules are usually less than about 2000 Daltons in size, alternatively less than about 1500, 750, 500, 250 or 200 Daltons in size, wherein such organic small molecules that are capable of binding, preferably specifically, to a wild type or mutant target protein may be identified without undue experimentation using well known techniques. In this regard, it is noted that techniques for screening organic small molecule libraries for molecules that are capable of binding to a polypeptide target are well known in the art (see, e.g., PCT Publication Nos. WO00/00823 and WO00/39585).

C. Antibodies

In some aspects, the agents for use in the methods described herein are antibodies. Antibodies are proteins that bind, preferably specifically, to a target protein. Variants of antibodies can be made based on information known in the art, without substantially affecting the activity of anti-body. For example, antibody variants can have at least one amino acid residue in the antibody molecule replaced by a different residue. For antibodies, the sites of greatest interest for substitutional mutagenesis generally include the hypervariable regions, but framework region (FR) alterations are also contemplated.

For antibodies, one type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino acid substitutions at each site.

D. Buffers

In some cases, the polypeptide composition further comprises a buffer solution. In some cases, the supported bilayers can be submerged in aqueous solution to prevent their destruction when they become exposed to air. The aqueous film and bulk liquid phase may be suitable aqueous solution, such as a buffered saline solution (e.g., PBS). The bulk solution can be readily changed by, e.g., flow-through rinsing with a solution having a different composition. In some cases, the buffer is selected from MES hydrate, MES monohydrate, BIS-TRIS, ADA, PIPES, ACES, MOPSO, BIS-TRIS propane, BES, MOPS, TES, HEPES, DIPSO, Trizma, TRICINE, GLY-GLY, EPPS, BICINE, TAPS, AMPD, AMPSO, CHES, CAPSO, AMP, CAPS, sodium bicarbonate, or TBS. In some cases, the buffer solution has a pH of at least 4, 5, 6, 7, 8, 9, or 10. In some cases, the buffer solution has a pH of at most 4, 5, 6, 7, 8, 9, or 10. In some cases, the buffer solution has a pH of 4, 5, 6, 7, 8, 9, or 10.

### IV. Ras Proteins and Fragments Thereof

Provided herein are compositions useful for identifying and detecting modulators of the conformational states of a protein complex comprising a Ras protein using second harmonic generation (SHG) technology. Also provided herein are methods for detecting a conformational change in the three-dimensional structure of a protein complex comprising Ras, wherein the protein complex is tethered to a cell-free lipid support layer.

A. Structure

G proteins such as Ras generally function as binary signaling switches with “on” and “off” states. In cells, these monomeric proteins generally cycle between a GDP-bound inactive state and GTP-bound active state. Ras protein or a fragment thereof can possess a six-stranded beta sheet and 5 alpha helices encompassing two main domains: a C domain (amino acids corresponding to amino acids 165-189 in SEQ ID NO. 1) and a G domain (amino acids corresponding to amino acids 1-165 in SEQ ID NO. 1). The C domain generally contains the CAAX motif capable of lipid-modification. The G domain of Ras is generally made up of five G motifs that bind GDP/GTP directly. In addition, Ras frequently contains two “switch” domains responsible for mediating the conformational change in the tertiary structure of the protein upon binding GTP or upon hydrolysis of GTP to GDP. “Switch 1” (amino acids corresponding to amino acids 32-38 in SEQ ID NO. 1) can generally contain threonine-35 of G2 while switch II (amino acids corresponding to amino acids 59-67 in SEQ ID NO. 1) can generally contain a critical glycine residue located in a DXXG motif. Ras protein or a fragment thereof can bind to a magnesium ion which assists in coordinating the binding of guanine nucleotide to the G domain. In the active GTP-bound state, the GTP γ-phosphate may interact with both the switch I and switch II regions of Ras, stabilizing the protein in a rigid conformation where effector proteins can bind and enable stimulatory signaling. Upon GTP hydrolysis, the switch I and II regions can relax into an inactive state that no longer binds effector proteins such as RAF1 and PI3 kinase. In cells, the process of exchanging the bound nucleotide can be facilitated by guanine nucleotide exchange factors (GEFs) and GTPase activating proteins (GAPs).

Native Ras proteins or a fragment thereof can contain a first region of 86 amino acids that is distinct to the Ras superfamily of proteins and can include the G-domain encompassing the Switch I and Switch II domain described above. The C-terminal sequence, known as the hypervariable region, frequently starts at amino acid 165 and can show low sequence similarity among Ras proteins except for a conserved CAAX motif (C, cysteine; A, aliphatic amino acid; X, methionine or serine) at the very C-terminal end, which is frequently present in all Ras proteins and can direct posttranslational processing.

B. Ras Proteins

Any Ras protein is contemplated for use in any of the methods disclosed herein. In some cases, the Ras protein can include H-Ras (NC_000011.10), K-Ras (including K-Ras 4A and K-Ras 4B) (NC_000012.12) and N-Ras (NC_000001.11), members of the Ras subfamily that are clinically notable as being implicated in many types of cancer. The three human Ras genes generally encode homologous proteins made up of chains of 188 to 189 amino acids, designated H-Ras, N-Ras and K-RAS4A and K-Ras 4B (the two K-Ras proteins arise from alternative splicing). In some aspects, the Ras protein can include other GTPase members from the Ras super family, for example, GTPases from the Rho, Rab, Ran and Arf small GTPase sub families. In some cases, the Ras protein can include many other members of the Ras subfamily, but are not limited to, DIRAS1, DIRAS2, DIRAS3, ERAS, GEM, MRAS, NKIRAS1, NKIRAS2, NRAS, RALA, RALB, RAPlA, RAP1B, RAP2A, RAP2B, RAP2C, RASD1, RASD2, RASL10A, RASLIOB, RASLI1A, RASL11B, RASL12, REM1, REM2, RERG, RERGL, RRAD, RRAS, RIT1, or RRAS2 (see also, Wennerberg et al., 2005, “The Ras superfamily at a glance,” J. Cell. Sci. 118 (Pt 5): 843-6, the disclosure of which is incorporated by reference in its entirety).

In some cases, the Ras protein can comprise the full-length Ras protein. In some cases, the Ras protein comprises a sequence as set forth in SEQ ID NO. 1.

In some cases, the Ras protein or fragment thereof includes the G-domain (amino acids corresponding to amino acids 1-165 in SEQ ID NO. 1). In some cases, the Ras protein or fragment thereof can lack the G-domain or a fragment thereof (amino acids corresponding to amino acids 1-165 in SEQ ID NO. 1). In some cases, the Ras protein or fragment thereof can include the C-domain (amino acids corresponding to amino acids 165-186 in SEQ ID NO. 1) or a fragment thereof. In some cases, the Ras protein or fragment thereof can include the C-domain or the G-domain or a combination of fragments thereof. In some cases, the Ras protein or fragment thereof can lack a switch I domain (amino acids corresponding to amino acids 32-38 in SEQ ID NO. 1) or a portion thereof. In some cases, the Ras protein or fragment thereof can lack a switch II domain (amino acids corresponding to amino acids 59-67 in SEQ ID NO. 1) or a portion thereof. In some cases, the Ras protein or fragment thereof can include the C-domain or a fragment thereof. In some cases, the Ras protein or fragment thereof, can include the CaaX motif at the C-terminus (corresponding to amino acids 184-186 in SEQ ID NO. 1) or a portion thereof. In some cases, the Ras protein contemplated herein can include a CC, CCX or CCXX motif.

In some cases, the Ras protein or fragment thereof can be a constitutively active Ras protein. In some cases, the Ras protein or fragment thereof can be a constitutively inactive Ras protein. In some embodiments, the mutant Ras protein or fragment thereof is unable to hydrolyze GTP. In other aspects, the mutant Ras protein or fragment thereof is unable to bind GDP. In some cases, the Ras protein or fragment thereof can be in a complex with GTP. In some cases, the Ras protein or fragment thereof can be in a complex with GDP. In some cases, the Ras protein or fragment thereof can be bound to a non-hydrolyzable GTP analog. In some cases, the GTP analog can be GppNHp. In some cases, the GTP analog can be modified with a bright, organic fluorophore (e.g., TAMRA, Cy3, or Cy5).

In some cases, the Ras protein or fragment thereof can include a post-translational modification. In some cases, the post-translational modification may occur at the C-terminus of the Ras protein or fragment thereof. In some cases, the post-translational modification may occur at the N-terminus of the Ras protein. In some cases, the Ras protein or fragment thereof can be modified by lipidation. In some cases, the lipid modifications include at least one of acylation, isoprenylation, N-myristoylation, S-palmitoylation, S-farnesylation or S-geranylgeranylation. In some cases, the lipidation can involve the addition of a myristate fatty acid. In some cases, the famesylation can include the addition of a 15-carbon famesyl isoprenoid chain. In some cases, the farnesylation can include the addition of a 20-carbon geranylgeranyl chain. In some cases, the Ras protein or fragment thereof can be modified at the C-terminus by proteolysis or carboxyl methylation. In some cases, the Ras protein or fragment thereof can be modified at the C-terminus by conditional modifications selected from phosphoryiation, peptidyI-prolyl isomerisation, monoubiquitylation, diubiquitylation, nitrosylation, ADP ribosylation and glucosylation.

In some cases, the Ras protein or fragment thereof can be in a complex with at least 1, 2, 3 or 4 proteins. In some cases, the effector protein can be a Raf kinase. In some cases, the effector protein can be selected from PI3K, RalGDS (NORElA), Af6, phospholipase C (PLC), Ras and Rab interactor 1 (RIN1), T cell lymphoma invasion and metastasis-inducing protein (TIAM), growth factor receptor 14 (Grbl4), and Bry2. In some cases, the effector protein can be Ras GEF protein. In some cases, the GEF protein can be selected from CNRASGEF, RASGEFlA, RASGRF2, RASGRP1, GRASGRP4, SOS1, RALGDS, RGL1, RGL2, RGR, C3G, PLC. In some cases, the effector protein can be GAP protein. In some cases, the GAP protein can be selected from NF1, IQGAP1, PLEXIN-B1, RASAL1, RASAl2, RAP1GAP, SIPA1 or TSC2.

In some cases, the Ras protein or fragment thereof can be a recombinant protein. In some cases, the recombinant Ras protein or fragment thereof can be produced in bacterial cells. In some cases, the recombinant Ras protein or fragment thereof can be produced in insect cell expression systems. In some cases, the recombinant bacterial cell or insect cell may be engineered to express processing enzymes for post-translational modification of a heterologous Ras protein or fragment thereof. In some cases, the enzymes that can process the post-translational modification can be selected from farnesyl transferase A, famesyl transferase B, CAAX prenyl protease 2, Protein-S-isoprenylcysteine O-methyltransferase, geranylgeranyltransferase II, geranylgeranyltransferase I and Rab geranylgeranyltransferase.

Also included in the disclosure are mutant forms of the Ras protein or fragment thereof. Oncogenic mutations in Ras proteins or fragments thereof can de-regulate cellular signaling by impairing GAP-mediated GTP hydrolysis, thus promoting tumorigenesis. In some aspects, the mutant Ras protein or fragment thereof can be a constitutively active Ras. As used herein, “constitutively active Ras” refers to a Ras protein which contains amino acid residue mutations that prevent the GAP-mediated hydrolysis of GTP, thus locking the Ras protein into a permanently “on” and active conformational state. In some embodiments, the Ras protein or fragment thereof can have one or more mutations at amino acid residue(s) G12, Q61, S17, or D119 in SED ID NO. 1. In some cases, the mutation can result in G12A/C/D/R/S/V, G13A/C/D/R/S/V or Q61H/K/L/P/R substitutions. In other embodiments, Ras can be mutated at amino acid residue number S17 or D119. Mutations at these residues (for example, S17N and D119N) can result in dominant negative Ras proteins.

In other aspects, the mutations in the Ras protein amino acid sequence prevent the protein or fragment thereof from adopting an active conformation (for example, the mutations result in a constitutively inactive Ras protein). In yet other aspects, the mutations in the Ras protein amino acid sequence or fragment thereof do not completely abolish the ability of the protein or fragment thereof to switch from one conformational state to the other but, rather, decreases or slows the ability of the protein or fragment thereof to do so. For example, in some embodiments, the mutation(s) slows the efficiency of the hydrolysis of GTP to GDP, thereby rendering the protein into a predominantly active conformational state. In some embodiments, the mutation(s) slows the hydrolysis of GTP by any of about 10%, 20%, 30%, 40%, 50%, 60%, 70%, 80%, or 100%, inclusive, including any values in between these percentages, in comparison to the hydrolysis of GTP in the wild type Ras protein. In another embodiment, the mutation(s) slows the efficiency of the binding of GTP into the empty active site of the Ras protein or fragment thereof. In some embodiments, the mutation(s) can slow the efficiency of the binding of GTP into the empty active site of the Ras protein or fragment thereof by any of about 10%, 20%, 30%, 40%, 50%, 60%, 70%, 80%, or 100%, inclusive, including any values in between these percentages, in comparison to the binding of GTP into the empty active site of the wild type Ras protein. In other embodiments, the mutation(s) inhibits the removal of GDP from the binding pocket of Ras protein or fragment thereof, thereby rendering the Ras protein into a predominantly inactive state. In some embodiments, the mutation(s) inhibit or slow the exchange of GDP for GTP by any of about 10%, 20%, 30%, 40%, 50%, 60%, 70%, 80%, or 100%, inclusive, including any values in between these percentages, in comparison to the exchange of these nucleotides in the wild type Ras protein.

In embodiments, the mutations in the Ras protein or fragment thereof can impair the protein from binding to other proteins. In embodiments, the mutations in the Ras protein or fragment thereof can prevent the protein from binding to effector signaling proteins. In embodiments, the mutations in the Ras protein or fragment thereof can prevent the protein from binding to Raf kinase proteins. In embodiments, the mutations in the Ras protein or fragment thereof can prevent the protein from binding to Raf kinase proteins. In some embodiments, the mutation(s) slows the efficiency of the binding of Ras protein or fragment to a native Raf protein or fragment thereof by any of about 10%, 20%, 30%, 40%, 50%, 60%, 70%, 80%, or 100%, inclusive, including any values in between these percentages, in comparison to the binding of a native Ras protein with a native Ras protein or fragment thereof.

In some cases, the mutant Ras proteins or fragments thereof can comprise oncogenic mutations. In some cases, exemplary mutations can include but are not limited to G12A/C/D/E/F/I/L/N/R/S/V/W/Y, G13A/C/D/E/I/N/R/S/V/, A11P/S/V, G10E, A11V, V7M, K5E/N, G15S/D, S17G, A18T, T20I, Q22K/R, A59E/G/T, V45A/I, T58I, A59E/G/T, G60A/D/V/E/R/, Q61D/E/H/K/L/P/R, E62D/K/G, V81M, A83D, K117E, D119N, E63, Y64N, S65C/R, R68T/S, L79I, D92N, A146T/P, K147N, R164Q, D69N, M72I, T74P, C80Y, E49K, C51Y, V141, S17G/N, A18D, L19F, T20S, L231, 124F, H27L, F28S, E31Q, P34S or T35I. References to particular amino acid residue numbers in the Ras protein primary structure above refer to the residue number corresponding to the aligned amino acid sequences of several Ras proteins. Alignment of Ras proteins for purposes of determining amino acid sequence residue number can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

### V. Raf Proteins

Provided herein are compositions useful for identifying and detecting modulators of the conformational states of a protein complex comprising a Raf protein using second harmonic generation (SHG) technology. In some embodiments, the protein complex is tethered to a cell-free lipid support layer. In some embodiments, the protein complex comprises Raf and Ras.

A. Structure

In mammals, the Raf family of serine/threonine kinases generally comprises members: Raf-1, A-Raf, and B-Raf, originating from three independent genes. Activation of Raf kinases frequently involves binding to activated Ras, dephosphorylation of inhibitory sites and phosphorylation of activating sites. Raf-1 generally contains a Ras-binding domain (RBD, aa 51-139), a cysteine-rich domain (CRD, aa 139-184), an ATP-binding motif (aa 355-363), a consensus serine/threonine kinase domain (aa 347-613) in the c-RAF sequence as set forth in SEQ ID NO. 3.

The three Raf isoforms (Raf-1, B-Raf, and A-Raf) can share a common modular structure consisting of 3 conserved regions (CR) with distinct functions. Conserved region 1 (CR1), generally contains a Ras-binding domain (RBD) that can interact with Ras, and a cysteine-rich domain (CRD), which can also contain a secondary Ras-binding site. The CRD can also help with the interaction of CR1 with the kinase domain for autoinhibition of Raf and with interaction of Raf with membrane phospholipids for membrane recruitment. CR2 can contain inhibitory phosphorylation sites participating in the negative regulation of Ras binding and Raf activation. CR3 can include the kinase domain, including the activation segment. Phosphorylation of the activation segment can lead to kinase activation.

Raf generally exhibits a typical kinase domain architecture, with the N-terminal lobe (N-lobe) and C-terminal lobe (C-lobe) linked through a short flexible hinge. The active site of the kinase can include a nucleotide (ADP or ATP) binding site, a magnesium binding site (DFG motif), and the phospho-acceptor site (activation segment). The motions between the lobes can be controlled by the flexible hinge and enable recruitment of the substrate and release of the product. The active conformation in Raf can be facilitated by dimerization.

B. Raf Proteins

In some cases, the Raf protein or fragment thereof contemplated herein can comprise the Ras binding domain (corresponding to amino acids 56-131 in SEQ ID NO. 3, amino acids 155-227 in SEQ ID NO. 4 or corresponding to amino acids 19-91 in SEQ. ID. NO. 5). In some cases, the Raf protein or fragment thereof can comprise the Ras binding domain and the cysteine-rich domain (corresponding to amino acids 138-184 in SEQ ID NO. 3, amino acids 234-280 in SEQ ID NO. 4 or corresponding to amino acids 98-144 in SEQ ID NO. 5). In some cases, the Raf protein or fragment thereof can comprise the Ras binding domain and the cysteine-rich domain or fragments thereof. In some cases, the Raf protein or fragment thereof can exhibit kinase activity corresponding to the kinase domain (corresponding to amino acids 349-609 in SEQ ID NO. 3, amino acids 451-717 in SEQ ID NO. 4 or corresponding to amino acids 310-570 in SEQ ID NO. 5). In some cases, the Raf protein or fragment thereof can comprise the Ras binding domain and the cysteine-rich domain and the kinase domain or portions thereof. In some cases, the Raf protein or fragment thereof can lack kinase activity. In some cases, the Raf protein can comprise a sequence as set forth in SEQ ID NO. 2. In some cases, the Raf protein can comprise a sequence that varies from the sequence set forth in SEQ ID NO. 2. In some cases, the Raf protein can comprise a sequence that has at least 70%, 80%, or 90% sequence identity to the sequence set forth in SEQ ID NO. 2.

In some cases, the Raf protein or fragment thereof contemplated herein can exhibit homodimerization or heterodimerization activity. In some cases, the Raf protein or fragment thereof contemplated herein can exhibit a lack of homodimerization or heterodimerization activity. In some cases, the Raf protein or fragment thereof can be bound to at least one zinc atoms. In some cases, the Raf protein or fragment thereof can be bound to at least one phospholipid. In some cases, the Raf protein or fragment thereof can be phosphorylated.

In some cases, the Raf protein or fragment thereof contemplated herein can comprise CR1 (corresponding to amino acids 51-194 in SEQ ID NO. 3, amino acids 150-290 in SEQ ID NO. 4 or corresponding to amino acids 14-154 in SEQ ID NO. 5) or fragments thereof. In some cases, the Raf protein contemplated herein can comprise the full-length c-Raf (SEQ ID NO. 3), full length-b-Raf (SEQ ID NO. 4) or full-length a-Raf (SEQ ID NO. 5) or fragments thereof. In some cases, the Raf protein or fragment thereof can comprise the CR2 region (corresponding to amino acids 254-269 in SEQ ID NO. 3, amino acids 360-375 in SEQ ID NO. 4 or corresponding to amino acids 209-224 in SEQ ID NO. 5) or portions thereof. In some cases, the Raf protein or fragment can comprise the CR3 (corresponding to amino acids 349-609 in SEQ ID NO. 3, amino acids 451-717 in SEQ ID NO. 4 or corresponding to amino acids 310-570 in SEQ ID NO. 5) or portions thereof. In some cases, the Raf protein contemplated herein can comprise CR1, CR2, CR3 or combinations of fragments thereof.

Also contemplated for use within the scope of the methods of the present disclosure are mutant forms of the Raf protein or a fragment thereof. In some cases, the mutation may be a point mutation. In some cases, the mutations may be present at amino acids corresponding to positions 427, 448, 461, 462, 463, 464, 465, 466, 467, 468, 469, 470, 478, 483, 581, 586, 593, 594, 595, 596, 597, 598, 599, 600, 601, 602 of B-Raf protein (SEQ ID NO. 4). Some exemplary mutations include E478K, S427G, I448V, V600E/K/D/R, R4621, 1463S, G464E/V/R, G469A/V/S, E586K, F595L, L597Q/R/S/V, A598V, T599I, K601E/N/T, A727V, G466A/E/V/R, S467A/E/L, G469E, K483M, N581I/S, D594A/E/G/H/N/V or G596A/C/D/R corresponding to amino acids in SEQ ID NO. 4. In some cases, the mutation may be a deletion of a portion of the Raf kinase (presented in SEQ ID NOs: 3, 4 or 5). References to particular amino acid residue numbers in the Raf protein primary structure can also refer to the residue number corresponding to the aligned amino acid sequences of B-Raf, A-Raf and C-Raf proteins. Alignment of Raf proteins for purposes of determining amino acid sequence residue number can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

In some cases, the mutation may reduce the activity of the Raf kinase compared to the wild-type protein. In some cases, the mutation may increase the activity of the Raf kinase compared to the wild-type protein. In some cases, the mutation can increase the activity of the mutant protein by up to 1, 3, 5, 7, 10, 12, 15, 17, 20, 25, 50, 60, 100, 150, 200, 250, 350, 500, 600, 700 or up to 1000-fold compared to the wild-type protein (e.g., V600E mutation). In some cases, the mutation can reduce the activity of the mutant protein by up to 1, 3, 5, 7, 10, 12, 15, 17, 20, 25, 50, 60, 100, 150, 200, 250, 350, 500, 600, 700 or up to 1000-fold compared to the wild-type protein (e.g., D594G mutation). In some cases, the mutation increases the kinase activity of the Raf kinase. In some cases, the mutation impairs the kinase activity of the Raf kinase. In some cases, the mutation can affect the dimerization activity of the Raf kinase (e.g., R509H in SEQ ID NO. 4). In some cases, the mutation can increase the dimerization activity of the Raf kinase or fragment thereof. In some cases, the mutation impairs the dimerization activity of the Raf kinase or fragment thereof.

In some cases, the Raf protein may be bound to an effector protein. In some cases, the effector protein can be a 14-3-3 protein. In some cases, the effector protein can be a kinase protein. In some cases, the effector protein can be a MAP kinase. In some cases, the effector protein can be MEK1, MEK2, MST2 or PKM. In some cases, the kinase protein can be Protein Kinase A or Protein Kinase B. In some cases, the effector protein can be c-Myc, IkB, Hsp90, Fyn, Cbl2, BAGI, b-subunit of trimeric G-protein, casein kinase, or Rheb.

In some cases, the complex contemplated herein can be a complex comprising a Raf kinase and a member of the Ras family. In some cases, the complex comprises A-Raf or a fragment thereof and K-Ras or a fragment thereof. In some cases, the complex can comprise N-Ras or a fragment thereof and B-Raf or a fragment thereof. In some cases, the complex can comprise H-Ras or a fragment thereof and B-Raf or a fragment thereof. In some cases, the complex can comprise K-Ras or a fragment thereof and B-Raf or a fragment thereof. In some cases, the complex can comprise Raf-1 or a fragment thereof bound to H-Ras, K-Ras or N-Ras or fragments thereof. In some cases, the complex can comprise Raf-1 or a fragment thereof bound to R-Ras3, Rit or TC21 or fragments thereof. In some cases, the complex can comprise Raf-1 or a fragment thereof bound to Rap1/2, Rin or Rheb proteins or fragments thereof in the Ras family.

In some cases, the Raf protein or fragment thereof may be bound to the Ras protein or fragment thereof using the RBD domain. In some cases, the Raf protein or fragment thereof may be in a complex with the Ras protein or fragment thereof via the CRD domain. In some embodiments, the Raf protein or fragment thereof may be membrane-bound via the CRD domain or fragment thereof. In some embodiments, the complex comprises a fragment of at least one of the proteins described above.

Methods for engineering a mutation or substitution into the primary amino acid sequence of a Ras or Raf protein are well known in the art via standard techniques. The Ras or Raf proteins for use in the methods described herein may include conservative substitutions. In some cases, substantial modifications in the biological properties of Ras or Raf proteins are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Non-conservative substitutions will entail exchanging a member of one of these classes for another class. Amino acids may be grouped according to common side-chain properties: (1) hydrophobic: Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe; (7) large hydrophobic: Norleucine, Met, Val, Leu, Ile;

In further embodiments, the mutant Ras or Raf proteins for use in the methods disclosed herein may comprise one or more non-naturally occurring or modified amino acids. A “non-naturally occurring amino acid residue” refers to a residue, other than those naturally occurring amino acid residues listed above, which is able to covalently bind adjacent amino acid residues(s) in a polypeptide chain. Non-natural amino acids include, but are not limited to homo-lysine, homo-arginine, homo-serine, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, omithine, citrulline, pentylglycine, pipecolic acid and thioproline. Modified amino acids include natural and non-natural amino acids which are chemically blocked, reversibly or irreversibly, or modified on their N-terminal amino group or their side chain groups, as for example, N-methylated D and L amino acids, side chain functional groups that are chemically modified to another functional group. For example, modified amino acids include methionine sulfoxide; methionine sulfone; aspartic acid-(beta-methyl ester), a modified amino acid of aspartic acid; N-ethylglycine, a modified amino acid of glycine; or alanine carboxamide and a modified amino acid of alanine. Additional non-natural and modified amino acids, and methods of incorporating them into proteins and peptides, are known in the art (see, e.g., Sandberg et al., (1998) J. Med. Chem. 41: 2481-91; Xie and Schultz (2005) Curr. Opin. Chem. Biol. 9: 548-554; Hodgson and Sanderson (2004) Chem. Soc. Rev. 33: 422-430).

In some embodiments, the mutant Ras or Raf proteins for use in the methods described herein can be isolated from cells (such as a cancer cell) by an appropriate purification scheme using standard protein purification techniques. In another embodiment, the mutant Ras or Raf proteins for use in the instant methods produced by recombinant DNA techniques. Alternative to recombinant expression, the mutant Ras or Raf proteins for use in the methods described herein can be synthesized chemically using standard peptide synthesis techniques.

In some examples, mutant target proteins for use in the methods described herein can be isolated from cells (such as a bacterial cell or an insect cell) by an appropriate purification scheme using standard protein purification techniques. In some examples, the purification techniques can be selected from ES-MS, immobilized metal affinity chromatography, etc. In another example, mutant target proteins for use in the instantly described methods are produced by recombinant DNA techniques. Alternative to recombinant expression, mutant target proteins for use in the methods described herein can be synthesized chemically using standard peptide synthesis techniques.

### VI. Conjugating Proteins to Supported Lipid Bilayers

A variety of methods are available for preparing a conjugate composed of a protein and a lipid bilayer. For example, water-soluble, amine-containing biomolecules can be covalently attached to lipids, such as phosphatidylethanolamine, by reacting the amine-containing biomolecule with a lipid which has been derivatized to contain an activated ester of N-hydroxysuccinimide.

As another example, biomolecules, and in particular large proteins, can be coupled to lipids according to reported methods. One method involves Schiff-base formation between an aldehyde group on a lipid, typically a phospholipid, and a primary amino acid on the protein. The aldehyde group is preferably formed by periodate oxidation of the lipid. The coupling reaction, after removal of the oxidant, is carried out in the presence of a reducing agent, such as dithiotreitol, as described by Heath, Biochem. et Biophys. Acta, 640:66 (1981). Typical aldehyde-lipid precursors suitable in the method include lactosylceramide, trihexosylceramine, galacto cerebroside, phosphatidylglycerol, phosphatidylinositol and gangliosides.

A second general coupling method is applicable to thiol-containing proteins and involves formation of a disulfide or thioether bond between a lipid and the protein. In the disulfide reaction, a lipid amine, such as phosphatidyl-ethanolamine, is modified to contain a pyridylditho derivative which can react with an exposed thiol group in the protein. Reaction conditions for such a method can be found in Martin, Biochemistry 20:4229 (1981). The thioether coupling method, described by Martin, J. Biol Chem. 257:286 (1982), is carried out by forming a sulfhydryl-reactive phospholipid, such as N-(4)P-maleimido-phenyl(butyryl)phosphatidylethanolamine, and reacting the lipid with the thiol-containing protein.

Another method for reacting a protein with a lipid involves reacting the protein with a lipid which has been derivatized to contain an activated ester of N-hydroxysuccinimide. The reaction is typically carried out in the presence of a mild detergent, such as deoxycholate.

Another method of linking proteins to a supported lipid bilayer is via specific interactions between the side chain of the amino acid histidine and divalent transition metal ions (Malik, et al, New J. Chem. 18:299-304 (1994); Arnold, Bio/Technol. 9:151-156 (1991)) immobilized on the membrane surface. This method has been used, for example, to attach various proteins and peptides to lipid monolayers (Shnek, et al., Langmuir 10:2382-2388 (1994); Frey, et al., Proc. Natl Acad. Sci. USA 93:4937 (1996)). Briefly, a cDNA encoding a protein (such as a ligand or receptor) which is immobilized to the bilayer surface is engineered so that the protein contains a poly-histidine (e.g., hexa-histidine) tag at one of its termini (e.g., the C-terminus). The bilayer is formed of or derivatized with metal-chelating moieties (e.g., copper-chelating moieties or lipids (Shnek, et al., Langmuir 10:2382-2388 (1994); Frey, et al., Proc. Natl Acad. Sci. USA 93:4937 (1996)), and the expressed His-tagged protein is incubated with the supported bilayer, or with the supported bilayer itself. In another embodiment, the supported lipid bilayer is nickelated and the expressed His-tagged protein is incubated with the supported bilayer.

Specific high-affinity molecular interactions may also be employed to link selected proteins to a supported bilayer. For example, a bilayer expanse may be formed to include biotinylated lipids (available from, e.g., Molecular Probes, Eugene, Oreg.), and a protein linked or coupled to avidin or steptavidin may be linked to the bilayer via the biotin moieties.

Proteins may also be linked to a supported lipid bilayer via glycan-phosphatidyl inositol (GPI). The proteins to be linked can be genetically engineered to contain a GPI linkage (Caras, et al., 1987; Whitehom, et al., 1995). Incorporation of a GPI attachment signal into a gene will cause the protein to be post-translationally modified by the cell resulting in a GPI linkage at the signal position. It will be appreciated that this type of alteration generally does not affect the molecular recognition properties of proteins such as the ones described here (McHugh, et al., Proc. Natl. Acad. Sci. USA, 92:8059-8063 (1995); Wettstein, et al., J. Exp. Med. 174:219-228 (1991).

In another aspect, any of the proteins disclosed herein can be attached to a supported lipid bilayer and labeled with an SH-active label (such as any of the SH-active labels disclosed herein) in situ.

### VII. Methods for Detecting a Conformational Change in Protein Complex Structure

A. Methods for Identifying Agents Capable of Inducing an Allosteric Change in the Structure of a Protein Complex.

In some aspects, provided herein are methods for identifying an agent that cause a conformational change in a polypeptide complex. In some embodiments, the method further comprises the steps of (a) contacting a polypeptide composition, any of which disclosed herein, (b) measuring an SHG signal from the SHG-active label, and (c) comparing the SHG signal to an SHG signal measured in an absence of the test agent, wherein a change in an amount of SHG signal in the presence of the test agent indicates that the test agent is an agent that causes a conformational change in the polypeptide complex. In some embodiments, the test agent contacts the first polypeptide and second polypeptide at an interface. In some embodiments, the test agent is a molecule with a molecular weight of less than 1000 AMU. In some embodiments, the method further comprises a change in the SHG-signal in the presence of the test agent. In some embodiments, the SHG-signal is increased by or by at least 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 300-fold, 400-fold, 500-fold, or 1000-fold. In some embodiments, the method further comprises treating cancer. In some embodiments, the method comprises administering a test agent to a subject in need thereof.

In some aspects, provided herein are methods for using SHG-based techniques to detect conformational changes within the three dimensional structure of a SH-labeled protein complex in conjunction with a supported cell-free lipid bilayer system. In some aspects, provided herein are methods for detecting a conformational change in the three dimensional structure of a protein complex (such as any of the protein complexes disclosed herein) bound to a supported lipid bilayer, wherein the protein complex is labeled with a second harmonic-active label (such as any of the SH-active labels disclosed herein), wherein the second harmonic-active label is hyperpolarizable, and wherein the second-harmonic label has a net orientation at an interface, the method comprising contacting the labeled protein complex with an agent, wherein the agent induces a conformational change in the three dimensional structure of the protein; and detecting light emitted from the interface using a surface selective technique so as to detect the conformational change in the three dimensional structure of the protein complex.

In some aspects, the method further comprises conjugating an SH-active label to a protein. In some cases, the SH-active label and protein are incubated together in a label: protein molar ration of about 2:1, 3:1, 4:1, 5:1, 6:1, 7:1, 8:1, 9:1 or 10:1 to generate an SH-active label conjugated protein. In some cases, the protein and SH-active dye can be incubated together for up to 0.1, 0.2, 0.3, 0.4, 0.5, 0.6, 0.7, 0.8, 0.9, 1, 1.1, 1.2, 1.6, 2.0, 2.5, 2.9, 3.3, 3.6, 3.8, 4.0, 4.5, 4.8, 5, 5.6, 6.0, 6.5, 6.9, 7.4, 7.8, 8.4, 8.9, 9.2, 9.5, or 10 hours. In some cases, the conjugation reaction can be terminated by removal of the SH-active. In some cases, the conjugation reaction can be terminated by exchange into protein storage buffer. In some cases, the conjugation reactions are conducted at room temperature. In some cases, the conjugation reactions can be conducted at 0° C., 1° C., 2° C., 3° C., 4° C., 5° C., 6° C., 7° C., 8° C., 9° C. or 10° C.

In embodiments, the ratio of SH-active label molecules (e.g., dye molecules) to the protein molecules to be labeled can affect the number of sites that are labeled. In embodiments, at least 30%, at least 40%, at least 50%, at least 60%, at least 70%, at least 80% or at least 90% or at least 99% of protein molecules in a conjugation reaction are labeled with the SH-active label. In some cases, the average degree of labeling (ratio of SH-active label to protein molecule is at least 0.5, 0.6, 0.7, 0.8, 0.9, 1.0, 1.1, 1.2, 1.3, 1.4, 1.5, 1.6, 1.7, 1.8, 1.9, 2.0, 2.2, 2.4, 2.5, 2.7, 3.0, 4, 5, 6, 7, 8, 9 or 10.

In embodiments, labeling can be performed specifically with respect to preselected locations on the protein. In some embodiments, protein labeling can be restricted to specific protein regions by modifying the labeling conditions. In some embodiments, labeling conditions such as dye-to-protein ratio, the pH, and the salt concentration can be modified. In some embodiments, the labeling products are analyzed by mass spectrometry to determine the reaction conditions that produce the desired product. In some embodiments, labeling can be restricted to a single specific residue on a target protein by site-specific engineering. For example, in some cases, the target protein can be engineered, for example, by site-directed mutagenesis to contain a single cysteine residue available for chemical conjugation.

In embodiments, the exogenous moieties (e.g., SH-active label) can be pre-attached to the protein in the protein complex, and any unbound or unreacted labels separated from the labeled entities before a surface-selective measurement is made. In one example, the second harmonic-active moiety (e.g., SH-active label) is attached to the protein (e.g., Ras or Raf proteins or fragments thereof) of the protein complex in vitro. In some examples, one or more constituent proteins (e.g. Raf protein) in the protein complex (e.g., complex comprising Ras or Raf proteins or fragments thereof) can be labeled with an SH-active moiety (e.g., dye) in situ (i.e., after being attached to a cell-free support surface). In other examples, one or more constituent proteins in the protein complex (e.g., complex comprising Ras or Raf protein or fragments thereof) are labeled with an SH-active moiety (e.g., dye) before being attached to a supported lipid bilayer surface or a supported lipid analog bilayer surface. In some examples, one or more labeling sites can be made more available for labeling for example by immobilizing the protein to be labeled on a lipid bilayer, thus exposing a portion of the protein that faces away from the lipid bilayer.

In some aspects, the method further comprises tethering a protein complex to a lipid-bi-layer. In some embodiments, provided herein are methods for tethering the protein complex on a lipid bi-layer, comprising the steps of mixing amounts of the first protein (e.g., Ras or a fragment thereof) and the second protein (e.g., Raf-1 or a fragment thereof), thereby forming said protein complex; and incubating said pre-formed protein complex on a lipid bilayer thereby tethering said protein complex to the lipid bilayer. In some embodiments, the method further comprises incubating the pre-formed protein complex for up to 1, 2, 3, 4, 5, 6, 7, 8, 9, 10, 11, 12, 14, 16, 18, 20, 22, 24, 26, 28, 30, 32, 36, 40, 44, 48, 52, 56, 60, 64, 68 or 72 hours with the lipid bi-layer. In some embodiments, the method further comprises washing the lipid bi-layer to remove excess unbound protein complex.

In other embodiments, provided herein are methods for tethering a protein complex on a lipid bi-layer, comprising the steps of tethering the first protein (e.g., Ras protein or fragment thereof) to the lipid bilayer and adding the second protein (e.g., Raf-1 protein or fragment thereof) to the first protein on the bilayer, thereby tethering the protein complex on the bilayer. In some embodiments, the method further comprises incubating the first protein for up to 1, 2, 3, 4, 5, 6, 7, 8, 9, 10, 11, 12, 14, 16, 18, 20, 22, 24, 26, 28, 30, 32, 36, 40, 44, 48, 52, 56, 60, 64, 68 or 72 hours in the lipid bi layer. In some embodiments, the method further comprises washing the lipid bi-layer to remove excess unbound first protein. In some embodiments, the method further comprises incubating the second protein with the first protein for up to 1, 2, 3, 4, 5, 6, 7, 8, 9, 10, 11, 12, 14, 16, 18, 20, 22, 24, 26, 28, 30, 32, 36, 40, 44, 48, 52, 56, 60, 64, 68 or 72 hours to form the complex in the lipid-bi layer. In some embodiments, the method further comprises washing the lipid bi-layer to remove excess unbound first protein. Further examples listing additional methods of forming protein-lipid conjugates are described in detail elsewhere in this disclosure.

In some aspects, provided herein are methods for detecting a conformational change in the structure of a protein complex comprising at least one SH-active label, and wherein the complex is tethered to a cell-free support layer. In embodiments, the complex is formed by binding of a Ras protein or fragment to a RAF kinase protein or fragment thereof. In embodiments, the Ras protein or a fragment thereof comprises an SH-active label. In embodiments, the Raf protein or a fragment thereof comprises an SH-active label. In embodiments, the method further comprises detecting a change in SHG signal response generated by the SH-label on the Ras protein using a surface-selective technique. In embodiments, the method further comprises detecting a change in SHG signal response generated by the SH-label on the Raf protein using a surface-selective technique. In embodiments, the change in SHG response signal indicates a conformational change in the protein complex.

In one aspect, provided herein are methods for identifying an agent that binds to an allosteric site on a constituent protein in a protein complex. In some cases, the protein complex is tethered to a cell-free support layer. In embodiments, provides herein are methods for identifying an agent that causes a conformational change in a protein complex comprising at least two proteins. In embodiments, the protein complex comprises Ras protein or a fragment thereof and a Raf protein or a fragment thereof. In embodiments, the method comprises: first, contacting the protein complex with the agent, wherein at least one of the proteins in the protein complex comprises an SH-active label. In some cases, the Ras protein or fragment thereof constituting the protein complex comprises an SH-active label. In some cases, the Raf protein or fragment thereof constituting the protein complex comprises an SH-active label.

In embodiments, the method further comprises comparing the amount of the detectable signal measured above, to an amount of a detectable signal measured in the absence of exposure of said protein complex to the agent, wherein a difference in the amount of the detectable signal indicates that the agent modulates the conformation of the protein complex. In some embodiments, the method further comprises incubating a first membrane-bound protein with an agent, wherein the agent can bind to the first protein to form a protein-agent conjugate, followed by addition of the second protein to the first protein-agent conjugate, wherein the first and second protein can form a protein complex bound to the agent. In other embodiments, the method further comprises adding a first protein to a membrane to form a membrane-bound protein, followed by addition of a second protein bound to an agent, wherein the first and second protein can then form a protein complex bound to the agent.

In embodiments, the method further comprises detecting a conformational change in a structure of the said protein complex upon binding of the agent to the protein complex by analyzing an amount of a detectable signal generated by the label using a surface selective technique. In some embodiments, the method includes detecting the detectable signal. In some embodiments, detecting the detectable signal includes measuring or quantifying an amount of the detectable signal. Detecting also includes detecting a presence or absence of a detectable signal. In embodiments, detecting can include detecting an absolute amount of a signal or a relative amount. In embodiments, the measurement is performed in arbitrary units. In some embodiments, the method includes activating the SH label. In some embodiments, the method includes combining photons interacting with a nonlinear material to form new photons with approximately twice the energy, and half the wavelength. In some embodiments, the method further includes, directing an intense laser beam (the fundamental) is directed to the interface of non-centrosymmetric sample, wherein the sample can generate nonlinear light, i.e. the harmonics of the fundamental. In embodiments, the method further includes detecting individual molecules or particles that possess hyperpolarizability (e.g., through the addition of a SH-active label) and are non-randomly oriented at a surface.

In some embodiments, the amount of a detectable signal measured in the presence of exposure of said protein complex above to the agent is compared to control conditions. For example, in some embodiments, the amount of a detectable signal measured in the presence of exposure of said protein complex above to the agent can be compared to the amount of a detectable signal measured in the absence of exposure of said protein complex to the agent.

In other embodiments, the amount of a detectable signal measured from the protein complex in the presence of the agent can be compared to a baseline signal response from the protein complex comprising at least a first protein and a second protein (e.g., Ras and Raf protein). In embodiments, the method further comprises the steps of comparing the detectable signal generated by a protein complex comprising at least two proteins tethered to a cell-free support layer, wherein the protein complex comprises at least one protein labeled with an SH-active label to (i) the detectable signal generated by the first protein tethered to the lipid bilayer without the second protein and to (ii) the detectable signal generated by the label attached to the second protein tethered to the lipid bilayer without the first protein. In other embodiments, the amount of a detectable signal measured from the protein complex in the presence the agent can be compared to a baseline signal response from the protein complex comprising at least a first protein and a second protein (e.g., Ras and Raf protein).

In embodiments, the method further comprises the steps of comparing the detectable signal generated by a protein complex comprising at least two proteins tethered to a cell-free support layer, wherein the protein complex comprises an SH-active label wherein one or more proteins in the protein complex is a wild-type protein to (i) the detectable signal generated by a protein complex comprising at least two proteins tethered to a cell-free support layer, wherein the protein complex comprises an SH-active label wherein at least one of the two proteins in the protein complex is a protein carrying a mutation (e.g., an oncogenic mutation).

In embodiments, the method further comprises measuring a detectable SHG signal from an SH-active labeled membrane-tethered protein composition comprising Ras and Raf protein or fragments thereof, wherein the Ras protein is in complex with GTP or an analog thereof, and comparing to the detectable SHG signal from the SH-active labeled membrane-tethered protein composition comprising Ras and Raf protein or fragments thereof, wherein the Ras protein is in complex with GDP or an analog thereof.

In embodiments, provided herein are methods for identifying an agent that causes a site-specific conformational change in the structure of a protein complex (e.g., comprising Ras and Raf proteins or fragments thereof). In embodiments, the method further comprises comparing (i) the first detectable signal generated by the protein complex (e.g., complex comprising KRAS and RafT protein) upon contacting with the agent, wherein the protein complex comprises a label at a first amino acid residue on a protein constituting the protein complex (e.g., Raf-1 protein to (ii) the second detectable signal generated by the protein complex upon contacting with the agent, wherein the protein complex comprises a label at a second amino acid residue on the constituent protein. In embodiments, the first amino acid is located in a different region of the constituent protein, as compared to the second amino acid. In embodiments, the first detectable signal is generated upon a conformational change in a region of the protein complex that is located in the vicinity of the first site and the second detectable signal is generated upon a conformational change in the protein complex at the second site, thereby providing a method for identifying an agent that causes a site-specific conformational change in the structure of a protein complex.

In other embodiments, provided herein are methods for identifying site-specific conformational changes in the structure of a protein complex comprising a Ras protein or a fragment thereof and a Raf protein or a fragment thereof. In some cases, the protein complex may consist of a Raf kinase or fragment thereof and a Ras protein or fragment thereof. In some aspects, the protein complex may consist of a Raf-1 kinase and a KRAS protein. In some cases, the protein complex comprising a Raf kinase and a Ras protein may comprise one or more SH-active labels attached to a first amino acid and/or a second amino acid in the Ras protein. In some cases, the protein complex comprising a Raf kinase and a Ras protein may comprise one or more SH-active labels attached to a first amino acid and/or a second amino acid in the Raf kinase protein. In some cases, the protein complex comprising a Raf kinase and a Ras protein may comprise one or more SH-active labels attached to a first amino acid and/or a second amino acid in the Ras protein and a first amino acid and/or a second amino acid in the Raf kinase protein. Methods for forming an SH-active labeled protein complex comprising Ras and Raf proteins or fragments thereof, are described elsewhere in this disclosure. In embodiments, the first detectable signal is generated upon a conformational change in a region of the protein complex that is located in the vicinity of the first amino acid label in a constituent protein (e.g., Raf-1 protein) and the second detectable signal is generated upon a conformational change in the protein complex at the second amino acid label in the constituent protein (e.g., Raf-1 protein), thereby providing a method for identifying an agent that causes a site-specific conformational change in the structure of a protein complex.

In embodiments, the method further comprises incubating the membrane-tethered SH-active labeled protein composition with an agent that can cause a conformation change in the structure of the protein composition (such as any of the candidate allosteric modulator agents described herein). In some cases, the protein composition comprises a protein complex. In some cases, the agent can induce the formation of the protein complex. In some cases, the agent can inhibit the formation of the protein complex. In some cases, the agent can cause dissociation of the pre-formed protein complex. In some cases, the agent can cause induce an allosteric change in the structure of the protein complex.

In some cases, the allosteric change can increase the activity of the protein complex by 1, 2, 5, 10, 20, 100 or 500-fold. In some cases, the allosteric change can decrease the activity of the protein complex by at least 2, 5, 10, 20, 100 or 500-fold.

In some cases, binding of an agent to the protein complex can cause a protein (e.g., Ras protein) in the protein complex (e.g., complex comprising Ras and Raf proteins or fragments thereof) to exhibit altered affinity to a ligand. In some cases, the allosteric change can cause a protein constituting the protein complex to exhibit increased affinity for a ligand by at least 2, 5, 10, 20, 100 or 500-fold. In some cases, the allosteric change can cause a protein constituting the protein complex to exhibit decreased affinity for a ligand by at least 2, 5, 10, 20, 100 or 500-fold. In some cases, the constituent protein can be KRAS. In some cases, the ligand can be GTP, GDP or analogs thereof. In some cases, the ligand can be an effector protein. In some cases, the effector protein can be a second protein constituting the protein complex. In some cases, the effector protein can be a RAF kinase. In some cases, the RAF kinase can be Raf-1.

In some cases, provided herein are methods for identification of agents that can cause a reduction in the oncogenic activity of a protein complex. In some cases, provided herein are methods for identification of agents that can cause a reduction in the oncogenic activity of a protein constituting a protein complex. In some cases, the protein complex comprises Ras protein or a fragment thereof and a Raf protein or a fragment thereof. In some cases, the agent can cause a reduction in the binding affinity of Ras protein to a Raf-1 protein, wherein the Ras and Raf-1 proteins together constitute a membrane-bound protein complex. In some cases, the agent can cause an increase in the binding affinity of Ras protein to a Raf-1 protein, wherein the Ras and Raf-1 proteins together constitute a membrane-bound protein complex. In some cases, the decrease in the binding affinity can indicate a reduction in the oncogenic activity of the protein complex. In some cases, the decrease in the binding affinity can indicate an increase in the oncogenic activity of the protein complex. In some cases, the decrease in the binding affinity can indicate an alteration in the downstream cellular signaling of the protein complex.

In embodiments, an SH-active labeled protein complex can be incubated with a candidate agent capable of causing an allosteric conformational change in the structure of the protein complex (such as any of the candidate allosteric modulator agents described herein). In embodiments, the agent can bind specifically to at least one of the constituent proteins in the protein complex. In some cases, binding of an agent to at least one of the constituent proteins in the protein complex can produce a change in the measurement of a detectable SHG signal. In some cases, a change in the measurement of a detectable SHG signal upon binding of an agent can constitute the presence or absence of a detectable SHG signal.

In some cases, the presence or absence of a detectable SHG signal upon binding of an agent can indicate a change in the conformational state of the protein complex. For example, the binding of an agent can cause a change in the measurement of a detectable SHG signal from a protein complex comprising at least one SH-active label. In some cases, the change in measurable SHG signal can include the loss of detectable SHG signal from a protein complex comprising at least one SH-active label. In some cases, loss of detectable SHG signal from a protein complex comprising at least one SH-active label upon binding of an agent can indicate dissociation of the constituent proteins in the protein complex. In some cases, the change in measurable SHG signal can include the appearance of a detectable SHG signal from a membrane-tethered protein composition comprising at least one SH-active label. In some cases, the appearance of a detectable SHG signal from a protein composition comprising at least one SH-active label upon binding of an agent can indicate the formation of a protein complex comprising the at least one SH-active label.

In some cases, the change in the measurable SHG signal upon binding of an agent can indicate an allosteric change resulting in a net change in orientation of the SH-active label attached to a constituent protein in the protein complex. In some cases, the change in the measurable SHG signal due to a net change in orientation of the SH-active label attached to a constituent protein in the protein complex can indicate a conformational change in the structure of the protein complex comprising the constituent protein.

In some aspects, binding of a candidate agent to a SH-active labeled protein complex can induce a conformational change in the structure of the target protein complex. In some examples, this conformational change can cause the net orientation of the SH-active label to change relative to the interface. In some examples, the net orientation of the SH-active label changes any of about 1°, 2°, 3°, 4°, 5°, 6°, 7° 8°, 9° 10°, or more relative to the interface upon binding to a candidate allosteric modulator agent. In one example, this change is detected and recorded in real time.

In embodiments, extremely subtle conformational changes, on the order of angstroms or sub-angstroms, can be measured in proteins bound to a surface which supports a lipid bilayer using SHG. In some embodiments, the root mean square standard deviation (RMSD) of the detected conformational change in the three dimensional structure of the protein is at least any of about 0.1 Å, 0.2 Å, 0.3 Å, 0.4 Å, 0.5 Å, 0.6 Å, 0.7 Å, 0.8 Å, 0.9 Å, 1 Å, 1.1 Å, 1.2 Å, 1.3 Å, 1.4 Å, 1.5 Å, 1.6 Å, 1.7 Å, 1.8 Å, 1.9 Å, 2 Å, 2.5 Å, 3 Å, 3.5 Å, 4 Å, 4.5 Å, 5 Å, 5.5 Å, 6 Å, 6.5 Å, 7 Å, 7.5 Å, 8 Å, 8.5 Å, 9Å, or 10Å or more, inclusive, including any numbers in between these values. In other embodiments the RMSD, is any of about 0.1Å to 1.6 Å, 0.2Å to 1.7Å A, 0.3Å to 1.8 Å, 0.4Å to 1.9 Å, 0.5Å to 2Å, 0.6Å to 2.1 Å, 0.7Å to 2.2 Å, 0.8Å to 2.3 Å, 0.9Å to 2.3 Å, 1Å to 2.4 Å, 1.1Å to 2.5 Å, 1.2Å to 2.6 Å, 1.3Å to 2.7 Å, 1.4Å to 2.8 Å, 1.5Å to 2.9 Å, 1.6Å to 3 Å, 1.7Å to 3.1 Å, 1.8Å, to 3.2 Å, 1.9Å to 3.3 Å, 2Å to 3.4 Å, 2.1Å to 3.5 Å, 2.2Å to 3.6 Å, 2.3Å to 3.7 Å, 2.4Å to 3.8 Å, 2.5Å to 3.9 Å, 2.6Å to 4 Å, 2.7Å to 4.1 Å, 2.8Å to 4.2 Å, 2.9Å to 4.3 Å, 3Å to 4.4 Å, 3.1 Å to 4.5 Å, 3.2Å to 4.6 Å, 3.3Å to 4.7 Å, 3.4Å to 4.8 Å, 3.5Å to 4.9 Å, 3.6Å to 5 Å, 3.7Å to 5.1 Å, 3.8Å to 5.2 Å, 3.9Å to 5.3 Å, 4Å to 5.5 Å, 4.5Å to 6Å, s A to 6.5 Å, 5.5Å to 7 Å, 6Å to 7.5 Å, 6.5Å to 8 Å, 7Å to 8.5 Å, 7.5Å to 9 Å, 8Å to 9.5 Å, 8.5Å to 10 Å, 9Å to 10.5 Å, 9.5Å to 11 Å, 10Å to 15 Å, 15Å to 20 Å, 20Å to 25Å, or 25Å to 30Å or more. In some embodiments, the conformational change in the three-dimensional structure of the protein complex is in an α-helical or β-sheet secondary structure of a constituent protein in the protein complex.

In some cases, the conformational change in the protein complex as measured by a change in the detectable SHG signal can result in a net change in the functional activity of the protein complex. In some cases, the conformational change in the protein complex as measured by a change in the detectable SHG signal can increase the activity of the protein complex by 1, 2, 5, 10, 20, 100 or 500-fold. In some cases, the allosteric change in the structure of a protein complex, indicated by a net change in measurable SHG signal can indicate a decrease the activity of the protein complex by at least 2, 5, 10, 20, 100 or 500-fold. In some cases, the activity of the protein complex can be measured as kinase activity. In some cases, the activity of the protein complex can be measured as protein-protein interaction activity. In some cases, the activity of the protein complex can be measured as cell signaling activity.

In some cases, the allosteric change indicated by a net change in orientation of the SH-active label attached to a constituent protein in the protein complex can cause a constituent protein (e.g., Ras protein) in the protein complex (e.g., complex comprising Ras and Raf proteins or fragments thereof) to exhibit altered binding affinity to a ligand. In some cases, altered binding affinity of the constituent protein to a ligand can involve an increase in binding affinity to the ligand. In embodiments, the binding affinity of the constituent protein to the ligand can be increased by at least 2, 5, 10, 20, 100 or 500-fold. In some cases, altered binding affinity to a ligand can involve a decrease in binding affinity to the ligand. In embodiments, the binding affinity of the constituent protein to the ligand can be decreased by at least 2, 5, 10, 20, 100 or 500-fold. In some cases, the constituent protein can be KRAS. In some cases, the ligand can be GTP, GDP or analogs thereof.

In some cases, provided herein are methods for identification of agents that can cause a reduction in the oncogenic activity of a protein complex. In some cases, provided herein are methods for identification of agents that can cause a reduction in the oncogenic activity of a protein constituting a protein complex. For example, in some cases, the allosteric change indicating by a net change in orientation of the SH-active label in a membrane-tethered protein complex can cause a constituent protein (e.g., Ras protein) in the protein complex (e.g., complex comprising Ras and Raf proteins or fragments thereof) to exhibit reduced oncogenic activity.

In some cases, the allosteric change indicated by a net change in orientation of the SH-active label attached to a constituent protein in the protein complex can cause a constituent protein (e.g., Ras protein) in the protein complex (e.g., complex comprising Ras and Raf proteins or fragments thereof) to exhibit altered binding affinity to a ligand. In some cases, the ligand can be an effector protein. In some cases, the effector protein can be a second constituent protein in the protein complex. In some cases, the effector protein can be a RAF kinase. In some cases, the RAF kinase can be Raf-1. In some cases, the effector protein can be a member of a Ras-regulated signaling pathway that control one or more of actin cytoskeletal integrity, cellular growth and/or proliferation, cellular differentiation, cell adhesion, apoptosis, or cell migration.

In some cases, the reduced oncogenic activity may be due to a change in the kinetics of association/dissociation of the two or more proteins constituting the SH-active labeled protein complex. In some cases, the reduced oncogenic activity may be due to a change in the affinity of a protein constituting the membrane-tethered SH-active protein complex to a ligand. In some cases, the change in affinity can be an increase in affinity of the constituent protein to the ligand. In some cases, the change in affinity can be a decrease in affinity of the constituent protein to the ligand. In some cases, the ligand can be a GTP, GDP or analogs thereof. In some cases, the ligand can be a protein. In some cases, the protein can be a second protein constituting the SH-active labeled protein complex. In some cases, the protein can be an effector protein involved in cell proliferation, cell survival, cell growth or cell migration.

In some cases, a change in the measurable SHG signal upon the binding of an agent as detected from a protein complex comprising Ras protein or a fragment thereof and a Raf protein and a fragment thereof, can indicate a reduction in the binding affinity of the Ras protein or fragment thereof to a Raf-1 protein or fragment thereof constituting the protein complex. In some cases, a change in the measurable SHG signal upon the binding of an agent as detected from a protein complex comprising Ras protein or a fragment thereof and a Raf protein and a fragment thereof, can indicate a reduction in the binding affinity of the Ras protein or fragment thereof to the Raf-1 protein or fragment thereof constituting the protein complex. In some cases, the decrease in the binding affinity can indicate a reduction in the oncogenic activity of the said protein complex. In some cases, the decrease in the binding affinity can indicate an increase in the oncogenic activity of the said protein complex.

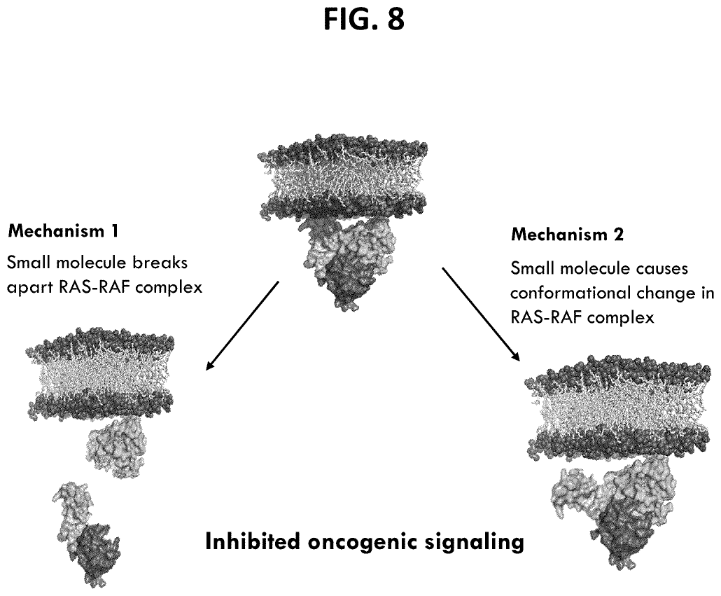

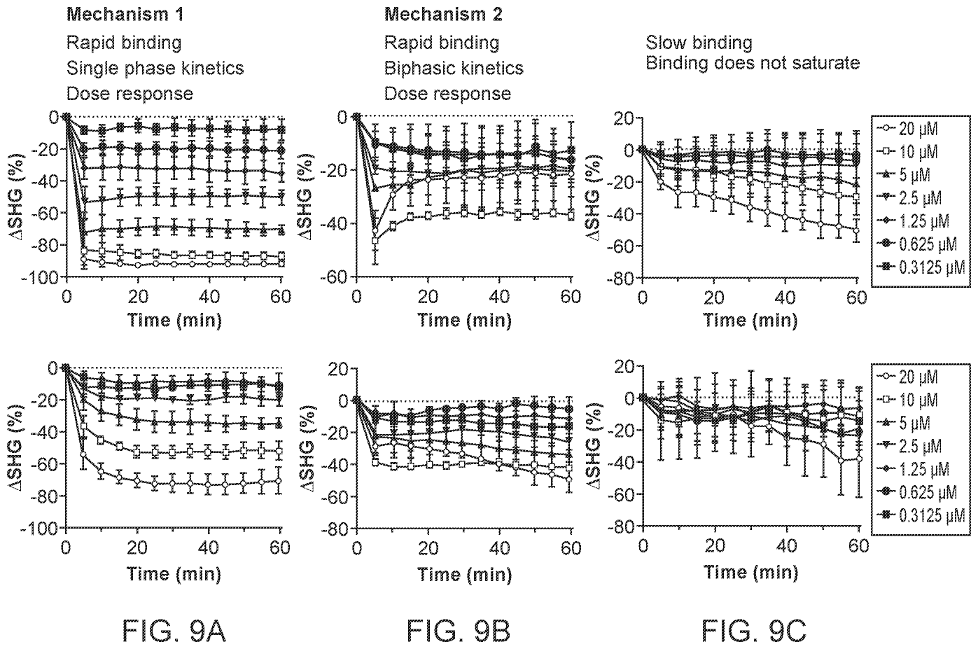

In one aspect, provided herein are methods for classifying the mechanism of action of an unknown agent by comparing it to the SHG signatures produced by other unknown ligands and classifying the binding class of the unknown ligands. In another aspect, provided herein are methods for classifying the mechanism of action of an unknown agent by comparing the SHG signature of the unknown agent to known SHG signatures obtained by binding of different known agents to a constituent protein in the protein complex, and classifying these to determine the binding class of the unknown ligands (for example, the signatures of unknown ligands can then be used to deduce the binding type (conformation produced) by comparing them to those of standard (known) drugs). For example, in some an agent may produce a signature SHG response, classified as mechanism 1, by causing the dissociation of a protein complex comprising Ras protein and Raf kinase as shown in FIG. 8 and FIG. 9A-C. In another example, an agent may produce a different signature SHG response, classified as mechanism 2, by causing a conformational change in a protein complex comprising Ras protein and Raf kinase as shown in FIG. 8B and FIG. 9A-C.

In some cases, provided herein are methods for measuring the specificity of binding of an agent to a tethered protein complex labeled with an SH-active label. In some cases, the detectable SHG signal produced by an SH-active labeled protein complex upon binding to a candidate agent, wherein the SH-active label located at a first site on a constituent protein can produce an altered SHG signal upon contacting the same candidate agent with the SH-active protein complex, wherein the SH-active label located at a second site on the constituent protein. In embodiments, the alteration in the measurable SHG signaling can indicate that the candidate agent binds specifically to the protein complex and induces a conformational change at the first site on the constituent protein and not at the second site. In some cases, the alteration in the measurable SHG signal can be a loss in the measurable intensity of the SHG signal when the SH-active label is located in the first site, but not at the second site. In some cases, the alteration in the measurable SHG signal can be a loss in the measurable intensity of the SHG signal when the SH-active label is located in the second site, but not at the first site. In some cases, a protein complex with an SH-active label located at a first site on a constituent protein and the protein complex with an SH-active label located at a second site on a constituent protein, can each produce a detectable signal upon incubation with a candidate allosteric modulator, indicating either that the candidate allosteric modulator is specifically binding to the protein complex and inducing a conformational change in the structure of the constituent target protein at both sites or that the candidate allosteric modulator is binding non-specifically to the constituent target protein.

In some cases, increasing the number of sites on the constituent target protein wherein SH-active labels are positioned can increase the ability to determine whether a candidate agent that can cause an allosteric conformational change binds specifically or non-specifically to a constituent target protein (i.e. the likelihood of a specific interaction decreases with the number of detectable signals produced over more than one, such as two, three, four or five, sites on a constituent target protein).

B. Generation of SHG Signal to Measure Conformational Changes in Protein Complex Structure.

Second harmonic generation (SHG) is a nonlinear optical process, in which photons interacting with a nonlinear material are effectively “combined” to form new photons with approximately twice the energy, and therefore twice the frequency and half the wavelength of the initial photons. It is a special case of sum frequency generation (SFG). Surface-selective nonlinear optical (SSNLO) techniques such as SHG allow the detection of interfacial molecules or particles in the presence of the bulk species. An intense laser beam (the fundamental) is directed on to the interface of some sample; if the interface is non-centrosymmetric, the sample is capable of generating nonlinear light, i.e. the harmonics of the fundamental. The fundamental or the second harmonic beams can easily be separated from each other, unlike the typical case in fluorescence techniques with excitation and emission light, which are separated more narrowly by the Stokes shift. Individual molecules or particles can be detected if they 1) are nonlinearly active (possess a hyperpolarizability) and 2) are near to the surface and through its influence (via chemical or electric forces) become non-randomly oriented. This net orientation and the intrinsic SHG-activity of the species are responsible for an SHG-allowed effect at the interface.

In embodiments, the method further comprises detecting a conformational change in a structure of the said protein complex upon binding of the agent to the protein complex by analyzing an amount of a detectable signal generated by the label using a surface selective technique. In some embodiments, the method includes detecting the detectable signal. In some embodiments, detecting the detectable signal includes measuring or quantifying an amount of the detectable signal. Detecting also includes detecting a presence or absence of a detectable signal. In embodiments, detecting can include detecting an absolute amount of a signal or a relative amount. In embodiments, the measurement is performed in arbitrary units. In some embodiments, the method includes activating the SH label. In some embodiments, the method includes combining photons interacting with a nonlinear material to form new photons with approximately twice the energy, and half the wavelength. In some embodiments, the method further includes, directing an intense laser beam (the fundamental) is directed to the interface of non-centrosymmetric sample, wherein the sample can generate nonlinear light, i.e. the harmonics of the fundamental. In embodiments, the method further includes detecting individual molecules or particles that possess hyperpolarizability (e.g., through the addition of an SH-active label) and are non-randomly oriented at a surface.

In embodiments, the method further comprises generating or delivering the nonlinear optical light (e.g., SHG) based on one or more of the following means: TIR (Total internal reflection), Fiber optics (with or without attached beads), Transmission (fundamental passes through the sample), Reflection (fundamental is reflected from the sample), scanning imaging (allows one to scan a sample), confocal imaging or scanning, resonance cavity for power build-up, multiple-pass set-up.

In embodiments, the method further comprises measuring the information in the form of a vector. In embodiments, the method further comprises measuring the vector in terms of one or more of the parameters selected from: intensity of light (typically converted to a photovoltage by a PMT or photodiode), wavelength of light (determined with a monochromator and/or filters), time, or position. In embodiments, the general configurations of the apparatus can include: image scanning (imaging of a substrate-intensity, wavelength, etc. as a function of x,y coordinate) and spectroscopic (measurement of the intensity, wavelength, etc. for some planar surface or for a suspension of cells, liposomes or other particles).

In embodiments, the method further comprises delivering the fundamental beam to the same. The fundamental beam can be delivered to the sample in a variety of ways (See, e.g., U.S. Patent Application Publication No.: 2002/0094528, the disclosure of which is incorporated by reference herein in its entirety). In embodiments, in sum- or difference-frequency configurations, the fundamental beams can be comprised of two or more beams, and can generate, at the interfaces, the difference or sum frequency beams.

### VII. Systems

Provided herein are systems for determining the conformational change induced in a protein complex (such as any of the complexes comprising Ras or Raf proteins described herein) by the binding of an agent. The system can have a substrate with a surface-attached protein complex comprising SHG-labeled Ras or Raf protein and an apparatus for generating and detecting a signal or signal change produced by the SHG-label upon the binding of an agent to the protein complex. The signal or signal change can be analyzed by the apparatus to produce a readout which is characteristic of the conformational change in the structure of the Ras or Raf protein that is induced by the agent.

In some embodiments, the system can have one or more of the following components: a source of a fundamental light, a substrate with a surface-attached SHG-labeled protein (for example, an SHG-labeled Ras or Raf protein) wherein the surface can be any of the surfaces described herein), a supported lipid bilayer, and a detector for measuring the intensity of the second harmonic or other nonlinear optical beams. The system can also employ, for example: a monochromator (for wavelength selection), a pass-filter, color filter, interference or other spectral filter (for wavelength selection or to separate the fundamental(s) from the higher harmonics), one or more polarizing optics, one or more mirrors or lenses for directing and focusing the beams, computer control, or software analyzing the detection signals correlated to the specific SHG-labeled protein (for example, an SHG-labeled Ras protein) or agent.

The apparatus for detection of Ras/Raf protein modulator interactions and their effects on conformational structure of the protein complex can assume a variety of configurations. In its most simple form, the apparatus can comprise the following: i) a source of the fundamental light; ii) a substrate with surface-attached probes (such as an SHG-labeled Ras or Raf); and iii) a detector for measuring the intensity of the second harmonic or other nonlinear optical beams. More elaborate versions of the apparatus can employ, for example: a monochromator (for wavelength selection), a pass-filter, color filter, interference or other spectral filter (for wavelength selection or to separate the fundamental(s) from the higher harmonics), one or more polarizing optics, one or more mirrors or lenses for directing and focusing the beams, computer control, or software.

According to another aspect, charge-coupled detectors (CCD) array detectors can be used when information is desired as a function of substrate location (x,y). CCDs comprise an array of pixels (i.e., photodiodes), each pixel of which can independently measure light impinging on it. For a given apparatus geometry, nonlinear light arising from a particular substrate location (x,y) can be determined by measuring the intensity of nonlinear light impinging on a CCD location (Q,R) some distance from the substrate—this can be determined because of the coherent, collimated (and generally co-propagating with the fundamental) nonlinear optical beam) compared with the spontaneous, stochastic and multidirectional nature of fluorescence emission. In embodiments, one or more array elements in the detector can map to specific regions of a substrate surface using a CCD array, allowing for easy determination of information as a function of substrate location (x,y). In embodiments, the method includes using photodiode detector and photomultiplier tubes (PMTs), avalanche photodiodes, phototransistors, vacuum photodiodes or other detectors known in the art for converting incident light to an electrical signal (i.e., current, voltage, etc.) can also be used to detect light intensities.

In embodiments, the CCD communicates with and is controlled by a data acquisition board installed in the apparatus computer. The data acquisition board can be of the type that is well known in the art such as a CIO-DAS16/Jr manufactured by Computer Boards Inc. The data acquisition board and CCD subsystem, for example, can operate in the following manner. The data acquisition board controls the CCD integration period by sending a clock signal to the CCD subsystem. In one embodiment, the CCD subsystem sets the CCD integration period at 4096 clock periods. By changing the clock rate, the actual time in which the CCD integrates data can be manipulated. During an integration period, each photodiode accumulates a charge proportional to the amount of light that reaches it. Upon termination of the integration period, the charge is transferred to the CCD's shift registers and a new integration period commences. The shift registers store the charges as voltages which represent the light pattern incident on the CCD array. The voltages are then transmitted at the clock rate to the data acquisition board, where they are digitized and stored in the computer's memory. In embodiments, the method further includes imaging a strip of the sample during each integration period. In embodiments, the method further comprises integrating a subsequent row until the sample is completely imaged.

In one aspect, the detector of the SH light can be a photomultiplier tube operated in single-photon counting mode. Photocurrent pulses can be voltage converted, amplified, subjected to discrimination using a Model SR445 Fast Preamplifier and Model SR 400 Discriminator (supplied by Stanford Research Systems, Inc.) and then sent to a counter. Photon counter gating and galvo control through a DAC output can be synchronized using a digital delay/pulse generator. Communication with a PC computer can be accomplished according to multiple methods as known to one skilled in the art, including but not limited to using a parallel register, a CAMAC controller card, and a PC adapter card.

In an alternative aspect, a bandpass, notch, or color filter is placed in either or all of the beam paths (e.g. fundamental, second harmonic, etc.) allowing, for example, for a wider spectral bandwidth or more throughput of light. In one embodiment of the methods provided herein, an interference, notch-pass, bandpass, reflecting, or absorbent filter can be used in place of the filters in the figures in order to either pass or block the fundamental or nonlinear optical beams.

In some aspects of the methods provided herein, the data recorded by the detector may be recorded on a fixed or data storage medium that is accessible via a system for reading the storage medium. For example, a system for reading a data storage medium may include a computer including a central processing unit (“CPU”), a working memory which may be, e.g., RAM (random access memory) or “core” memory, mass storage memory (such as one or more disk drives or CD-ROM drives), one or more display devices (e.g., cathode-ray tube (“CRT”) displays, light emitting diode (“LED”) displays, liquid crystal displays (“LCDs”), electroluminescent displays, vacuum fluorescent displays, field emission displays (“FEDs”), plasma displays, projection panels, etc.), one or more user input devices (e.g., keyboards, microphones, mice, touch screens, etc.), one or more input lines, and one or more output lines, all of which are interconnected by a conventional bidirectional system bus. The system may be a stand-alone computer, or may be networked (e.g., through local area networks, wide area networks, intranets, extranets, or the internet) to other systems (e.g., computers, hosts, servers, etc.). The system may also include additional computer-controlled devices such as consumer electronics and appliances.

Input hardware may be coupled to the computer by input lines and may be implemented in a variety of ways. In one aspect of the methods herein, the machine-readable data of this disclosure may be inputted via the use of a modem or modems connected by a telephone line or dedicated data line. Alternatively, or additionally, the input hardware may include CD-ROM drives or disk drives. In conjunction with a display terminal, a keyboard may also be used as an input device.

In another embodiment, the method includes coupling the output hardware to the computer by output lines and implementing by conventional devices. By way of example, the output hardware may include a display device for displaying a graphical representation of an active site of this disclosure using a program such as QUANTA. Output hardware might also include a printer, so that hard copy output may be produced, or a disk drive, to store system output for later use.

Machine-readable storage devices useful in the present disclosure include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage of data.

A person having skill in the art will appreciate that any other method or technique to communicate or store data is also contemplated for providing real time data of Ras or Raf family protein conformational changes upon binding a candidate modulator in a machine-readable format.

SHG has emerged as a sensitive technique to detect and study the conformational changes of biomolecules using SH-active probes (Salafsky, J. S. Journal of Chemical Physics 2006, 125, 074701; Salafsky, J. S. Physical Chemistry Chemical Physics 2007, 9, 5704). Labeled proteins that are adsorbed or covalently immobilized on surfaces produce an SHG signal, which is due to the average, net orientation of the nonlinear polarizability of the SHG label relative to the surface plane. Specifically, the SH intensity is given as I_{SH}=G(χₛ⁽²⁾)²I², where I_{SH} is the second-harmonic intensity, G is a constant that depends on the experimental geometry and wavelength, and I is the intensity of the fundamental beam. The nonlinear susceptibility, χₛ⁽²⁾, carries the details of the SH-active molecules on the surface via the equation:

χₛ^{(2)m}Nₛ<α⁽²⁾>, where Nₛ is the surface density of the molecules, the brackets denote an orientational average, and α⁽²⁾ is their nonlinear polarizability, a quantum-mechanical property that determines the probability of producing a second-harmonic photon from two, incident photons of the fundamental beam. Measurements of χₛ⁽²⁾ provide information about the orientation of a molecule on the surface. For example, when α⁽²⁾ is dominated by a single element ζζζ⁽²⁾ along the molecular axis ζ and the azimuthal distribution of the molecules are random in the plane of the surface, the only elements of χₛ⁽²⁾ that do not vanish are:

χ_{s⊥⊥⊥}⁽²⁾=Nₛ<cos³θ>α_{ζζζ}⁽²⁾

χ_{s⊥∥∥}⁽²⁾=χ_{s∥⊥∥}⁽²⁾=χ_{s∥∥⊥}⁽²⁾=½Nₛ<cos θ sin²θ>α_{ζζζ}⁽²⁾

where θ is the polar angle between and the surface normal, and the subindices L and I refer to the directions perpendicular and parallel to the surface, respectively (Heinz, T. F., et al., Physical Review A 1983, 28, 1983).

The SH light is coherent and directional, so collection and isolation of the SH beam is simplified, and because the fundamental and the second-harmonic are well separated spectrally, cross-talk, which can plague fluorescence measurements, is non-existent with SHG. Photodegradation of the probe occurs relatively slowly via two-photon-induced absorption, allowing measurements over relatively long timescales. The trade-off with SHG is signal strength—it is orders of magnitude weaker than fluorescence. However, only SH-active molecules immobilized on the surface contribute second harmonic light since randomly diffusing molecules near the surface produce no signal; their orientational average, from Equation 1, is zero. Therefore, SHG is intrinsically equipped to discriminate between surface-bound and free molecules. The SH signal reports on the orientational average of the probes, and thus changes due to conformational change.

### IX. Kits

Also provided herein are kits for use in performing any of the methods disclosed herein. The kit may include one or more of 1) any of the surfaces or interfaces described herein for immobilizing or attaching a protein (for example, a Ras or Raf protein), 2) any of the SH-active labels described herein for labeling a protein (for example, a Ras or Raf protein), 3) any of the apparatuses for eliciting an second harmonic signal or signal change described herein, 4) any of the apparatuses for analyzing the signal or signal change, wherein the analyzed signal indicates whether an agent has altered the conformational structure of a protein (for example, a Ras or Raf protein), and/or 5) any of the surfaces or interfaces described herein for detecting a conformational change in the three dimensional structure of a protein (for example, a Ras or Raf protein) bound to a supported lipid bilayer.

### VIII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “in vitro” is used to describe an event that takes places contained in a container for holding laboratory reagent such that it is separated from the biological source from which the material is obtained. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein “second harmonic” refers to a frequency of light that is twice the frequency of a fundamental beam of light.

As used herein, a molecule or material phase is “centrosymmetric” if there exists a point in space (the “center” or “inversion center”) through which an inversion (x,y,z)->>(−x,−y,−z) of all atoms is performed that leaves the molecule or material unchanged. A non-centrosymmetric molecule or material lacks this center of inversion. For example, if the molecule is of uniform composition and spherical or cubic in shape, it is centrosymmetric.

Centrosymmetric molecules or materials have no nonlinear susceptibility or hyperpolarizability, necessary for second harmonic, sum frequency and difference frequency generation.

As used herein, “surface-selective” refers to a non-linear optical technique such as second har-monic generation or sum/difference frequency generation or other surface-specific technique known in the art.

As used herein, “sum frequency generation” (SFG) is a nonlinear, optical technique whereby light at one frequency (Ω1) is mixed with light at another frequency (Ω2) to yield a response at the sum frequency (Ω1+Ω2) (Shen, 1984, 1989). For example, SFG is particularly useful for the detection of molecules at surfaces through their characteristic vibrational transitions and, in this case, is essentially a surface-selective infrared spectroscopy with Ω1 and Ω2 at visible and infrared frequencies. When the terms “SHG” or “second harmonic generation” are used herein, it is understood that SFG and “sum frequency generation” can substitute and be used in place of SHG with methods well known to one skilled in the art.

A “nonlinear active moiety,” as used herein, is a substance which possesses a hyperpolarizability.

“Second harmonic-active moiety” or “second harmonic-active moiety,” as used herein, refers to a nonlinear-active moiety, particle or molecule which can be attached (covalently or non-covalently) to a molecule (e.g., a protein, such as an enzyme), particle or phase (e.g., lipid bilayer) in order to render it more nonlinear optical active.

“Allosteric”, “allosteric modulator”, or “allosteric candidate” as used herein, refers to a molecule, moiety or substance which binds predominantly to a site other than the active site and causes conformational change as determined by SHG or SFG, and thus exert their effect via an allosteric mechanism of action.

“Active site” or “active binding site,” as used herein, refers to a region of a target protein that, as a result of its shape and charge potential, favorably interacts or associates with another agent (including, without limitation, a ligand, a protein, polypeptide, peptide, nucleic acid, including DNA or RNA, molecule, compound, antibiotic or drug, molecule, moiety, substrate, product, analog, or inhibitor) via various covalent and/or non-covalent binding forces and where function of the protein is performed such as, but not limited to, catalysis, signaling, and/or effector activation.

“Hyperpolarizability” or “Nonlinear Susceptibility” as used herein refer to the properties of a molecule, particle, interface, or phase which allow for generation of nonlinear light. The terms “hyperpolarizability,” “second-order nonlinear polarizability,” and “nonlinear susceptibility” are some-times used interchangeably.

As referred to herein, sites that participate in a binding partner event which are “functionally relevant”, as defined herein, includes any sites which make direct or indirect structural contact with the binding partner (e.g., effector molecule) as determined by a structural technique such as X-ray crystallography, NMR or SHG. Direct structural contact is defined as any residue, some portion of which is within 2 nm of some portion of the binding partner molecule. Indirect structural contact is defined as any residue, some part of which changes its orientation, conformation or relative coordinates upon binding of binding partner (e.g., effector molecule), or a binding partner mimic or analog, as seen by a structural technique such as X-ray, NMR or SHG, relative to its orientation, conformation or relative coordinates in the absence of the binding partner, mimic or analog. The term “functionally relevant” also includes residues which are known to be important in the binding or the modulation (e.g., activation, inhibition, regulation, and so on) of the binding molecule by a non-structural means (e.g., mutagenesis or biochemical data which shows that particular residues are important for binding or modulation of the binding partner).

The term “ligand”, as defined herein includes any molecule that binds to another molecule, such as, but not limited to, one protein binding to another, a carbohydrate binding to a protein, or a small molecule binding to a protein.

As used herein, “nonlinear” refers to optical techniques capable of transforming the frequency of an incident light beam (a.k.a., the fundamental). The nonlinear beams are the higher order frequency beams which result from such a transformation, e.g. a second harmonic. In second harmonic, sum frequency or difference frequency generation, the nonlinear beams are generated coherently. In second harmonic generation (SHG), two photons of the fundamental beam are virtually scattered by the interface to produce one photon of the second harmonic. Also referred to herein as “nonlinear optical” or “surface-selective nonlinear.”

The terms “nonlinear active” or “nonlinearly active” as used herein also refer to the general proper-ty of the ability of molecules, particles, an interface or a phase, to generate nonlinear optical radiation when driven by incident radiation beam or beams.

When referring herein to nonlinear optical methods, “detection” or “detecting” refers to those techniques by which the properties of surface-selective nonlinear optical radiation can be used to detect, measure or correlate properties of probe-target interactions (such as the interaction be-tween a protein and a candidate modulator compound), or effects of the interactions, with properties of the nonlinear optical light (e.g., intensity, wavelength, polarization or other property common to electromagnetic radiation).

As used herein, the term “conformational change” refers to the alteration of a biological species' (for example, a protein, such as an enzyme) structural conformation.

As used herein, the term “protein” includes polypeptides, peptides, fragments of polypeptides, and fusion polypeptides.

As used herein, the term “modulator” refers to any substance (e.g., small molecule compound, peptide, protein, etc.) which alters the conformation of a protein as detected by SHG.

As used herein, an “interface” is a region which generates a nonlinear optical signal or the region near a surface in which there are second harmonic-active moiety-labeled targets possessing a net orientation. An interface can also be composed of two surfaces, a surface in contact with a different medium (e.g., a glass surface in contact with an aqueous solution, a cell surface in contact with a buffer), or the region near the contact between two media of different physical or chemical properties. An interface can also be composed of two polypeptide surfaces in contact with one another.

As used herein, a “mutation” includes an amino acid residue deletion, an amino acid residue insertion, and/or an amino acid residue substitution of at least one amino acid residue in a defined primary amino acid sequence, such as a primary amino acid sequence of a target protein. An amino acid “substitution” means that at least one amino acid component of a defined primary amino acid sequence is replaced with another amino acid (for example, a cysteine residue or a lysine residue). Desirably, mutation or substitution of one or more amino acid residues (such as a conservative mutation or substitution) in a primary amino acid sequence does not result in substantial changes in the susceptibility of a target protein encoded by that amino acid sequence to undergo a conformational change upon binding to a ligand of that target protein or upon binding to an unknown candidate agent capable of allosterically binding a target protein. Methods for engineering a mutation or substitution into the primary amino acid sequence of a protein such as a target protein are well known in the art via standard techniques.

Unless defined otherwise herein, all technical and scientific terms used herein have the same meaning as commonly understood by one of ordinary skill in the art to which this disclosure pertains.

As used herein, the singular terms “a,” “an,” and “the” include the plural reference unless the con-text clearly indicates otherwise.

It is intended that every maximum numerical limitation given throughout this specification includes every lower numerical limitation, as if such lower numerical limitations were expressly writ-ten herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein

The section headings used herein are for organizational purposes only and are not to be construed as limiting the subject matter described

### IX. Examples

The following examples are included for illustrative purposes only and are not intended to limit the scope of the disclosure.

### Example 1: Preparation of SHG-Active RAF1^{RBDCRD} by Conjugation of SHG Probe to RAF1

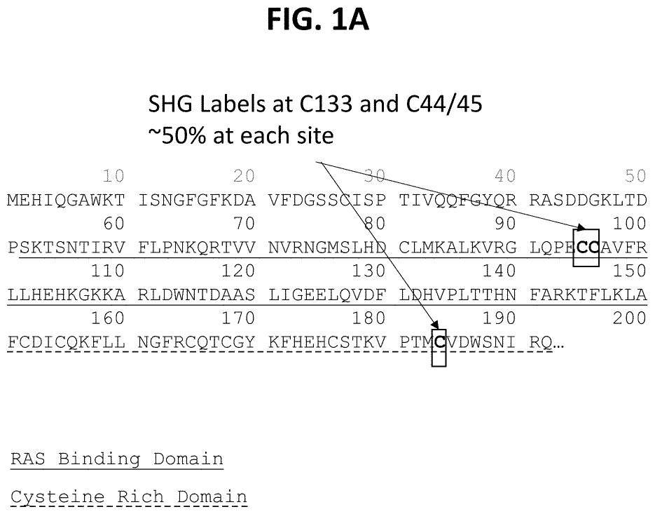

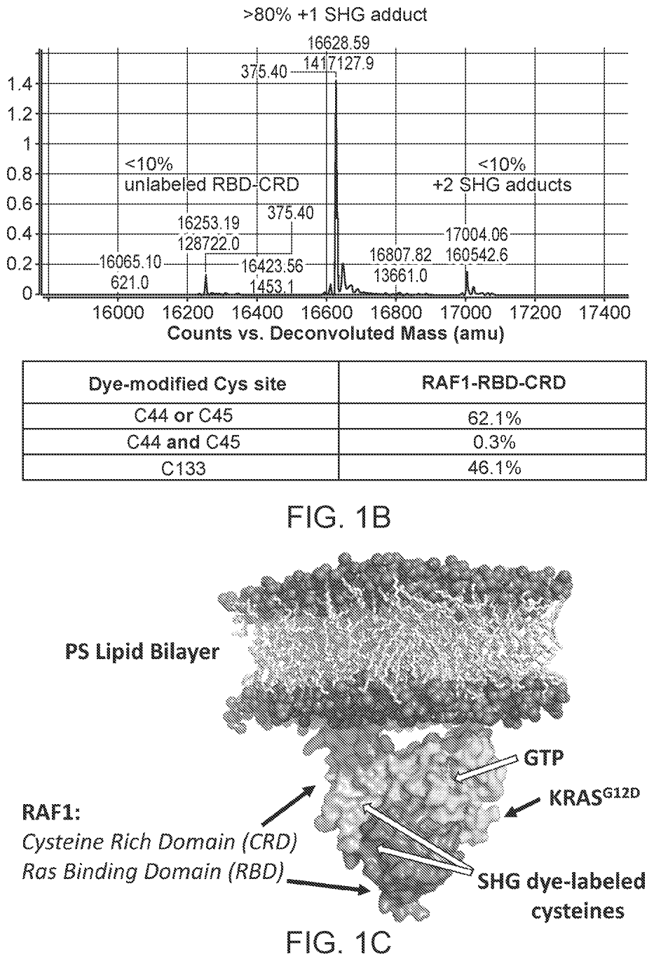

SHG-active RAF1^{RBDCRD} conjugates were prepared by dye conjugation to a lysine-reactive (SHG1) probe or to a cysteine-reactive (SHG2) probe (FIG. 1A). 30 μM conjugation reactions were carried out in the presence of 2× and 5× dye: protein molar ratios to achieve biologically active SHG-active RAF1^{RBDCRD}. Specifically, the SHG1 reaction was carried out in sodium bicarbonate, pH 8.3 buffer for 10 min on ice and the SHG2 reaction was carried out in sodium phosphate, pH 6.6 buffer for 90 min on ice. Conjugation reactions were terminated by dye removal and exchange into protein storage buffer, and UV-Vis analysis was used to measure the degree of labeling (DoL) defined as the average ratio of dye molecules to protein molecules. Both the SHG1 and SHG2 probes successfully conjugated such that a 5×SHG1 reaction produced RAF1^{RBDCRD}-SHG1 with 0.9 DoL and a 2×SHG2 reaction produced RAF1^{RBDCRD}-SHG2 with 1.1 DoL.

Mass spectrometry peptide analysis of this conjugate revealed that RAF1^{RBDCRD}-SHG2 contains a dye-modified residue at amino acids corresponding to C44, C45 or C133 in SEQ ID NO. 2 (amino acids corresponding to C45, C95 or C184 respectively in SEQ ID NO. 3) (FIG. 1B). Mass spectrometry analysis of RAF1RBDCRD-SHG2 showed that the majority (greater than 80%) of this protein conjugate carries one dye molecule per protein molecule (FIG. 1B). These residues are located near the site of interaction between KRAS and RAF1 (FIG. 1C), enabling capture of conformational changes near the effector interface. This labeling allows the monitoring of conformational changes of the target complex near the protein-protein interaction site with the goal of identifying ligands to the complex or ligands that affect the conformation of this protein-protein interaction.

### Example 2: Formation of KRAS^{G12D}-FMe+SH-Active RAF1^{RBDCRD} Complex on a Phosphatidyl Serine Bilayer

A. Preparation of Lipid Bilayer

The phosphatidylserine (PS)-enriched bilayer was prepared from PS-enriched small unilamellar vesicles (SUVs) purchased from Biodesy, Inc. The concentrated SUVs were diluted in TBS buffer containing 5 mM CaCl₂) and incubated on a glass-bottom 384-well Biodesy Delta™ assay plate to allow bilayer formation. The bilayer surface was washed repeatedly with assay buffer to remove excess lipid.

B. Formation of Protein Complex

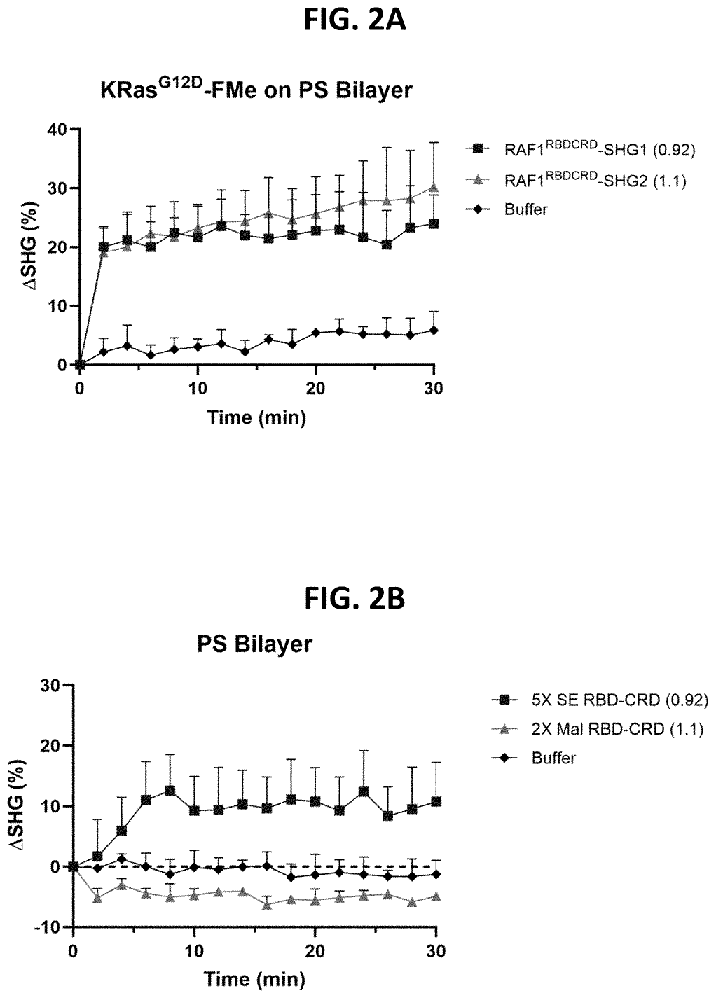

KRAS only binds RAF1 when KRAS is in its active GTP bound conformation. To maintain this state in experiments, KRAS bound to the non-hydrolyzable analog, GppNHp, was used. GppNHp-bound KRAS^{G12D}-FMe was tethered to the PS-enriched lipid bilayer surface overnight at 4° C. The formation of the complex of KRAS^{G12D}-FMe+RAF1^{RBDCRD} complex was monitored by SHG. Briefly, RAF1 SHG conjugates were injected onto phosphatidylserine (PS)-enriched bilayer in the presence or absence of tethered KRAS^{G12D}-FMe bound to the non-hydrolyzable GTP analog, GppNHp. The following day untethered protein was removed and SHG intensity was monitored upon injection of RAF1^{RBDCRD} conjugates (FIG. 2A). As a control, RAF1^{RBDCRD} conjugates were also injected onto PS bilayer alone (FIG. 2B).

C. Measurement of SHG Signal with Protein Complex Formation

Bound RAF1 was expected to produce a detectable SHG signal distinct from the baseline bilayer signal because SHG conjugates produce a signal dependent on orientation.

Since SHG conjugates produce a signal dependent on orientation; therefore, only bound RAF1 was expected to produce a detectable SHG signal distinct from the baseline bilayer signal. Both RAF1^{RBDCRD}-SHG1 and RAF1^{RBDCRD}-SHG2 produced a detectable increase in SHG intensity upon RAF1 injection indicative of binding to tethered KRAS^{G12D}-FMe plateauing at about 30 minutes (FIG. 2A) in the presence of GppNHp bound KRAS^{G12D}-FMe. RAF1 conjugates were also monitored upon addition to PS bilayer without tethered KRAS^{G12D}-FMe to ensure that the SHG signal change observed upon RAF1 addition was due to interaction with KRAS, (FIG. 2B). Interestingly, RAF1^{RBDCRD}-SHG1 (0.9) produced a 10% signal change on bilayer alone, suggesting some nonspecific interaction between the RAF1 conjugate and the PS surface. In contrast, RAF1^{RBDCRD}-SHG2 had no effect on the PS surface in the absence of KRAS.

### Example 3: Tethering of Pre-Formed KRAS^{G12D}-FMe+SH-Active RAF1^{RBDCRD} Complex on a Phosphatidyl Serine Bilayer

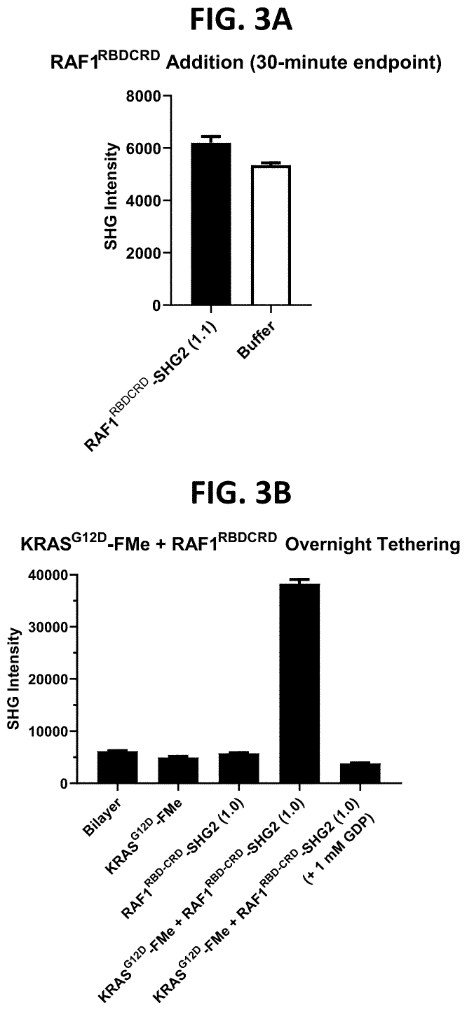

The increase in SHG intensity noted after addition of SH-active RAF1^{RBDCRD}-SHG2 to the tethered KRAS^{G12D}-FMe and subsequent formation of KRAS^{G12D}+RAF1 complex formation in real-time was observed to be only about 1000 counts larger than the buffer control injection (FIG. 3A). Factors contributing towards maximizing the complex SHG signal compared to bilayer-tethered KRAS^{G12D}-FMe alone were further optimized in order to develop a robust screening assay that would allow the separation of real conformational modulators from the baseline signal noise. Accordingly, a pre-formed complex was tethered to the bilayer and the baseline SHG intensity of the complex was compared to each individual component. Equimolar amounts of KRAS^{G12D}-FMe and RAF1^{RBDCRD}-SHG2 were mixed and incubated together on a PS bilayer overnight, in order to monitor the ability to capture the pre-formed complex on a PS bilayer for conformational detection.

RAF1^{RBDCRD}-SHG2 conjugates were tested for their ability to a) produce a measurable SHG signal upon association with lipid-anchored KRAS^{G12D}-FMe and b) produce a measurable SHG signal change upon complex dissociation. In comparison, each protein component was incubated on the PS bilayer to make sure the SHG signal generated by the complex was distinct from any signal produced by the individual components (FIG. 3B). The PS bilayer alone produced a baseline SHG signal around 6000. As expected, both KRAS^{G12D}-FMe and RAF1^{RBDCRD}-SHG2 produced SHG signals similar to bilayer alone. Since KRAS^{G12D}-FMe was tethered to the membrane via its farnesyl tail, but carries no SHG probe, no SHG signal above bilayer was expected. Similarly, while RAF1 carries the SHG probe, it only becomes orientated on the surface through its interaction/binding with active KRAS. By contrast, the pre-formed complex was expected to produce a significant SHG signal above 30,000 counts (FIG. 3B). To determine that this complex signal was due to the biologically relevant protein interaction, the complex was also tethered overnight in the presence of 1 mM GDP (FIG. 3B). Over time, the excess GDP would compete with the GppNHp bound to KRAS, pushing KRAS into its inactive state that can no longer bind RAF1. In the presence of GDP, no complex SHG signal was detected, demonstrating that the complex forms in a GTP-dependent manner (FIG. 3B).

### Example 4: Optimization of Buffer Conditions for Development of Robust Screening Assay of KRAS^{G12D}-FMe+SH-Active RAF1^{RBDCRD} Interaction on Phosphatidyl Serine Bilayer

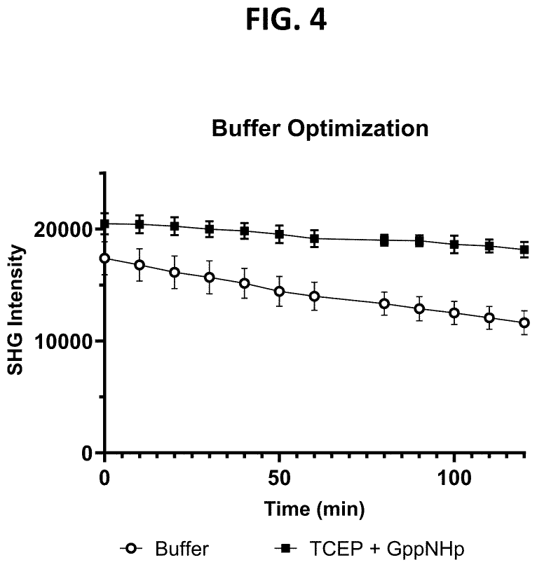

The signal stability from the active KRAS^{G12D}-FMe+RAF1^{RBDCRD}-SHG2 complex on a PS bilayer by SHG was monitored over time. In the presence of assay buffer (20 mM Hepes, 150 mM NaCl, 1 mM MgCl2, pH 7.4), the baseline signal showed a substantial 30% decrease over two hours (FIG. 4). In order to minimize signal loss due to RAF1^{RBDCRD}-SHG2 dissociation over time, it was necessary to stabilize the KRAS^{G12D}-FMe+RAF1^{RBDCRD}-SHG2 complex interaction. To stabilize the KRAS^{G12D}-FMe+RAF1^{RBDCRD}-SHG2 complex interaction, the assay buffer was supplemented with 1 mM TCEP to prevent cysteine oxidation and 5 μM GppNHp was added to prevent nucleotide dissociation from KRAS^{G12D}-FMe. These were added to the assay buffer for overnight protein tethering as well as the protein wash the following day. The addition of both TCEP and GppNHp increased both the baseline signal and stabilized it significantly such that only 12% signal loss was detected over two hours (FIG. 4). Stabilization of the interaction allowed development of a robust screening assay with minimal baseline change over time for identification of true conformational modulators.

### Example 5: Optimization of Positive Control Conditions for Screening Assay of KRAS^{G12D}-FMe+SH-Active RAF1^{RBDCRD} Interaction on Phosphatidyl Serine Bilayer

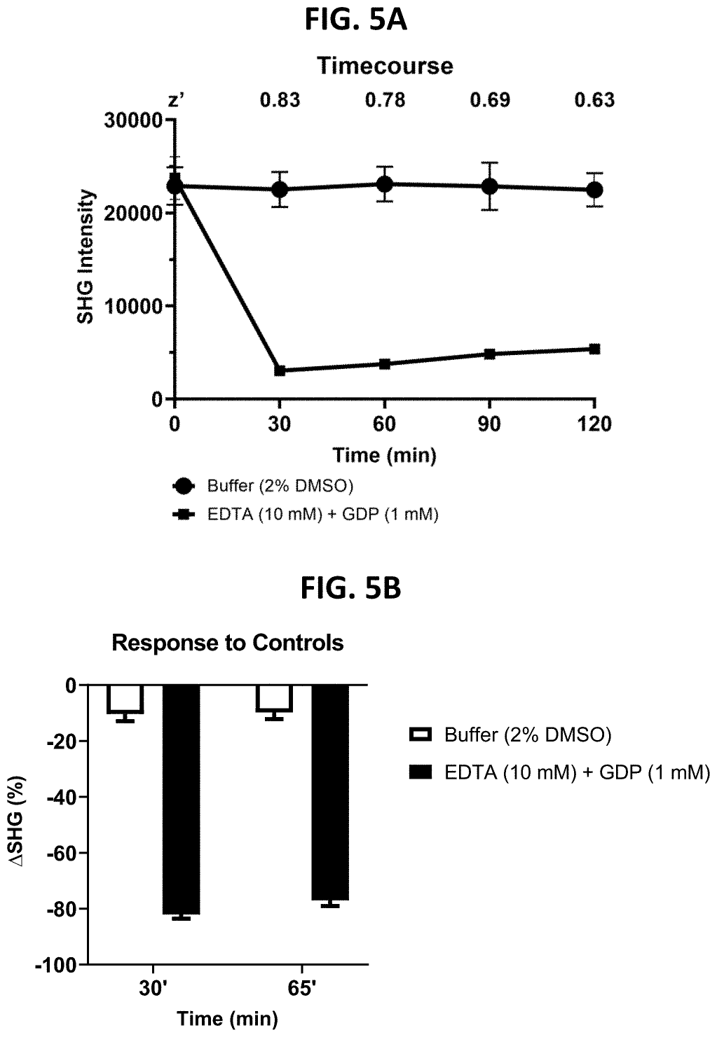

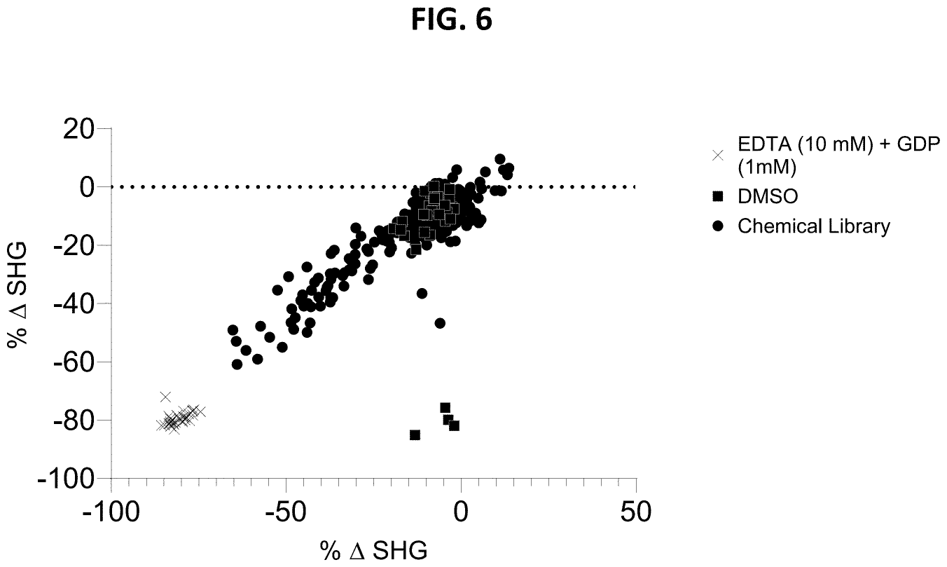

In order to validate the system, it was necessary to identify a positive control that is consistent with known biology and shows a well-characterized and measurable signal change over an ample dynamic range to allow separate statistically significant hits from assay noise. Since RAF1 binding to KRAS is nucleotide dependent, the SHG signal change was monitored in the presence of 10 mM EDTA and 1 mM GDP. EDTA, a metal chelator causes GppNHp dissociation from KRAS and excess GDP, thus promoting complex dissociation, which was observed as a robust decrease in signal intensity by about 80% in the first 30 minutes and maintained over at least 120 minutes (FIG. 5A and FIG. 5B). The SHG response to this control reinforced that the detected complex signal behaves according to its characterized biology. Additionally, the well-differentiated response to controls gave a Z′ score of 0.8, which indicated that the assay conditions were suitable for high throughput screening (FIG. 6).

### Example 6: Optimization of Counter-Screen Assay Conditions for Screening Assay of KRAS^{G12D}-FMe+SH-Active RAF1^{RBDCRD} Interaction on Phosphatidyl Serine Bilayer

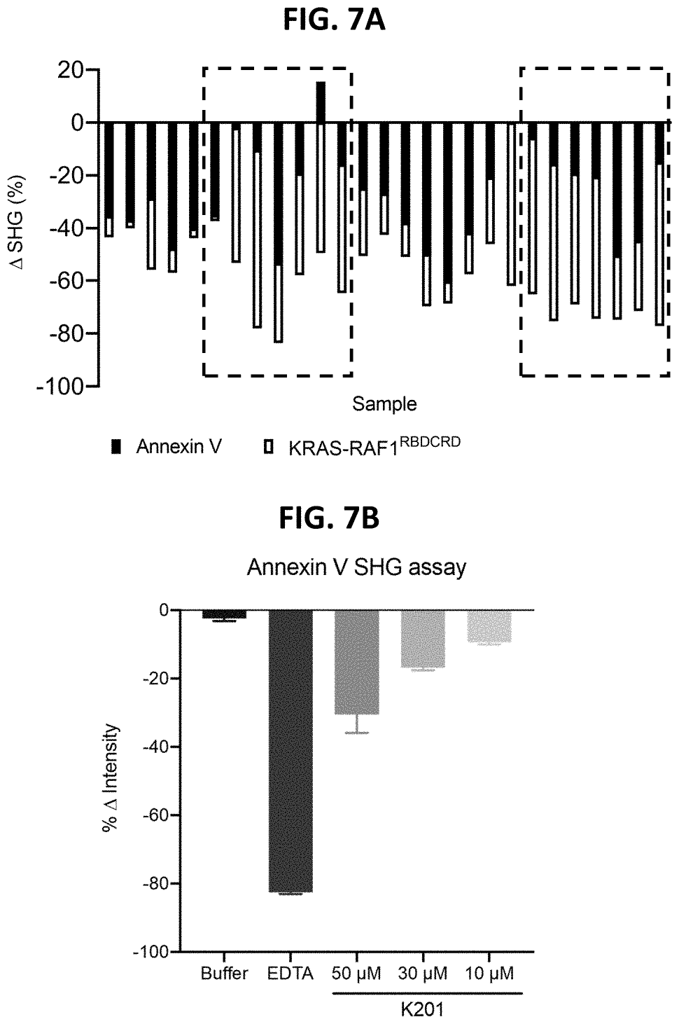

Annexin V binds to phosphatidyl serine membrane like KRAS^{G12D}-FMe in a calcium-dependent manner. However, Annexin V is structurally unrelated to KRAS/RAF1 complex. Addition of EDTA, a chelation agent, results in >80% decrease in SHG signal due to loss of Annexin V protein from the phosphatidyl serine surface. A dose-dependent SHG response was also observed upon addition of a control small molecule ligand K201 (Kaneko N et. al, 1997) to Annexin V (FIG. 7B). This assay was established as a counter-screen assay to the KRAS^{G12D}-FMe+SH-active RAF1^{RBDCRD} interaction, allowing detection of false-positives due to metal chelation or non-specific protein binding. As shown in FIG. 7A, compounds that passed primary screening criteria were assayed against Annexin V, and only compounds that produced a significant and reproducible SHG response against KRAS^{G12D}-FMe+SH-active RAF1^{RBDCRD} (blue) compared to Annexin V (red) were retained for further characterization (within green boxes).

### Example 7: SHG-Based Detection of KRAS^{G12D}-FMe+SH-Active RAF1^{RBDCRD} Interaction on Phosphatidyl Serine Bilayer

A beam from a Ti:S femtosecond laser is used as the fundamental according to procedures known to those skilled in the art. Specifically, an argon-pumped Ti:Sapphire system operating at 80 MHz with ^{˜}150 fs pulse duration and 0.5 W average power was employed (Coherent, Inc.). The beam is preferentially focused to a spot at the slide-buffer interface. Second harmonic light generated by the surface is collected, filtered from the fundamental, and detected by a photomultiplier tube (PMT) according to procedures known to those skilled in the art. A baseline signal with declining intensity due to photobleaching is recorded. The polarization of the fundamental beam is varied to produce the maximum signal output. The polarization of both the fundamental and second-harmonic beams is varied using wave plates. The signal is verified as the second-harmonic by determining its quadratic dependence on the fundamental intensity and measuring its characteristic spectral line shape. Each data point is obtained by using a photon counting 1-second integration time.

### Example 8: SHG-Based Detection of Multiple Mechanistic Profiles of KRAS^{G12D}-FMe+SH-Active RAF1^{RBDCRD} Interaction on Phosphatidyl Serine Bilayer

Real-time change in the SHG signal upon ligand addition produces kinetic profiles that allow classification and differentiation of conformational signatures produced by ligand binding. For example, in the KRAS^{G12D}_FMe+SH-active RAF1^{RBDCRD}SHG assay, addition of EDTA+GDP (positive control), produced a rapid dose-dependent signal decrease due to chemical-induced complex dissociation. Some ligands produced a similar signature with a rapid, stabilized signal decrease characteristic of complex dissociation. These ligands were grouped together as “Mechanism 1” (FIG. 8, 9A).

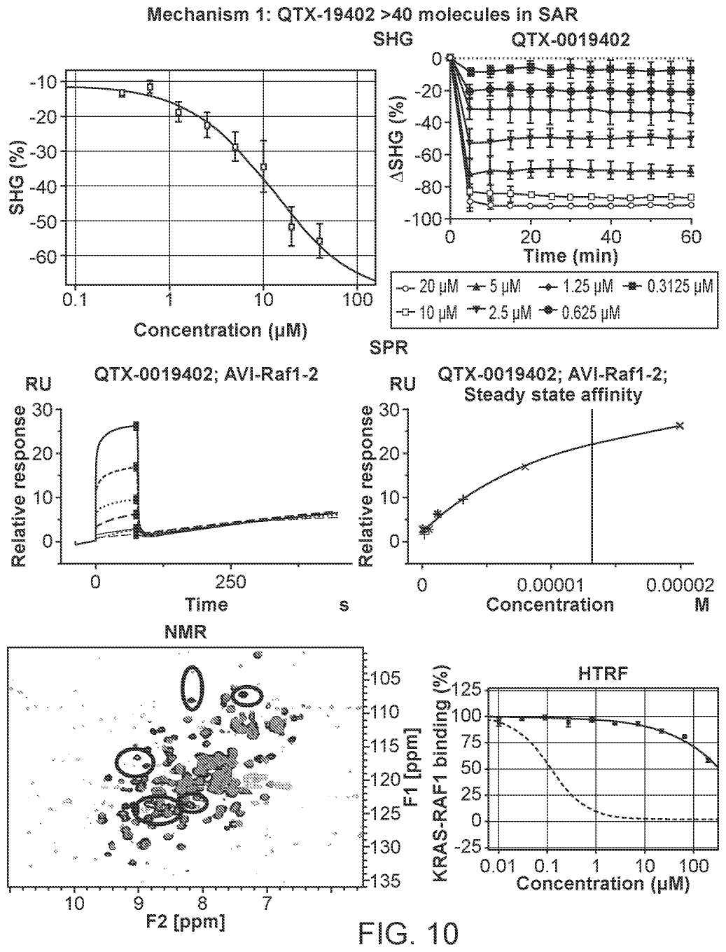

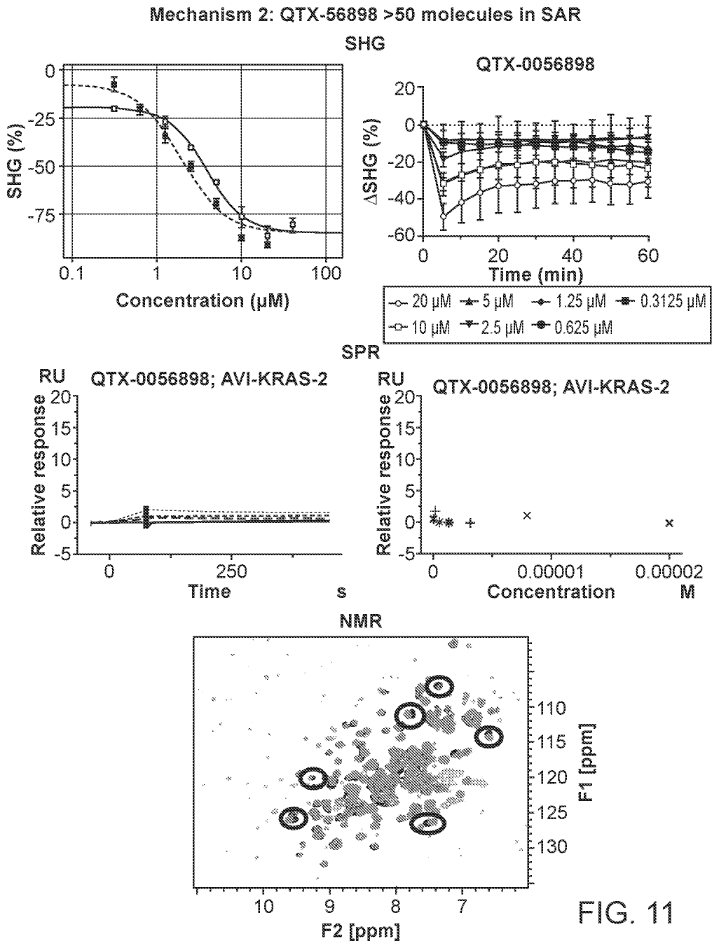

Alternatively, some ligands produced an initial ligand binding event, which caused an immediate conformational change followed by a slower rearrangement that stabilizes in the presence of ligand. These ligands produced a distinct biphasic kinetic signature where an immediate signal decrease was followed by a slight signal increase that stabilized, mimicking the biphasic response previously observed by SHG and other techniques in proteins upon binding of allosteric modulators (Donohue et al., 2019). Conformational signatures produced by small molecules are therefore classified based on the kinetics of the SHG signal change produced upon binding to the protein complex (FIG. 9A, FIG. 9B and FIG. 9C). These novel mechanisms of action were further confirmed using orthogonal structural methods such as SPR, NMR or X-Ray crystallography (FIG. 10 and FIG. 11).

### Example 9: Screening a Small-Molecule Library for Agents that Modulate the Binding of Ras to Raf Protein in a Membrane Tethered Complex

Equimolar amounts of KRAS^{G12D}-FMe and RAF1^{RBDCRD}-SHG2 are mixed and incubated together on a phosphatidyl-serine (PS) bilayer overnight to allow tethering to the lipid membrane. Next day, excess unbound protein complex is washed off to retain only the tethered protein complex on the membrane. Each protein component is incubated on the PS bilayer to make sure the SHG signal generated by the complex is distinctly higher than any signal produced by the individual components. The assay buffer is supplemented with 1 mM TCEP to prevent cysteine oxidation and 5 μM GppNHp to prevent nucleotide dissociation from KRAS^{G12D}-FMe. Stabilization of the interaction by addition of TCEP and GppNHp increases both the baseline signal and stabilizes it significantly to allow development of a robust screening assay with minimal baseline change over time.

In a parallel experiment, the complex is tethered overnight and the change in the SHG signal change is monitored in the presence of 10 mM EDTA and 1 mM GDP. EDTA, a metal chelator causes GppNHp dissociation from KRAS and excess GDP in the presence of 1 mM GDP, leading to the formation of an inactive KRAS conformation that can no longer bind to RAF1.Robust decrease of SHG signal in the presence of GDP (at least 80% loss of signal intensity in the first 30 minutes after addition to the complex) is used to confirm that the signal from the complex is due to biologically relevant protein interaction. Counter-screen conditions are established by monitoring the SHG response of Annexin V to EDTA or to different concentrations of a control small molecule ligand K201. Annexin V allows for false-positives detection, such as non-specific protein binders or chelating agents, because Annexin V binds to phosphatidyl serine membranes and is structurally unrelated to KRAS/RAF1 complex. As shown in FIG. 7A, ligands that produced a significant and reproducible SHG response against KRAS^{G12D}-FMe+SH-active RAF1^{RBDCRD} (blue) were assayed against Annexin V (red) before being retained for further characterization (within green boxes). Both the primary and counter-screen assay conditions are optimized to establish a well-differentiated response to control conditions, generating a Z′ score of 0.8 that is suitable for high throughput screening,

Agents from a small-molecule library (for example, a marine-derived natural product library) are contacted with the Ras/Raf protein complex in a high-throughput manner. For example, Ras/Raf protein complexes are tethered to a lipid analog bilayer in buffer (for example, a phosphatidylserine-based lipid bi-layer) in microwells supported by a glass surface. Small molecules from the library are contacted using a high-throughput robotic interface in the microwells containing the protein complex in assay buffer. Multiple positive control conditions (Ras/Raf protein complex in buffer containing EDTA and excess GDP) and negative control conditions (assay buffer) are included in each assay plate to establish a proper Z′ score for identification of true-positive and true-negative allosteric modulators in the assay.

Next, SHG responses are detected using a beam from an argon-pumped Ti:sapphire system operating at 80 MHz with ˜150 fs pulse duration and 0.5 W average power (Coherent, Inc.). The beam is preferentially focused to a spot at the slide-buffer interface. Second harmonic light generated by the surface is collected, filtered from the fundamental, and detected by a photomultiplier tube (PMT) according to procedures known to those skilled in the art. A baseline signal with declining intensity due to photobleaching is recorded. The polarization of the fundamental beam is varied to produce the maximum signal output. The polarization of both the fundamental and second-harmonic beams is varied using wave plates. The signal is verified as the second-harmonic by determining its quadratic dependence on the fundamental intensity and measuring its characteristic spectral line shape. Each data point is obtained by using a photon counting 1-second integration time.

Different SHG “signatures” (SHG responses) upon binding of different known agents to the KRAS^{G12D}-FMe+SH-active RAF1^{RBDCRD} complex are classified by grouping similar signatures (kinetic and endpoint measurements) and comparing the signatures to the control conditions, as described in Example 8.

The identified agents are confirmed as candidate cancer therapeutics with further study. Similar methods can be used to screen peptides and antibodies or other novel types of agents that can modulate the binding of Ras and Raf in a membrane tethered protein complex.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCES

| SEQ | | |
|---|---|---|
| ID# | SEQUENCE | ANNOTATION |
| 1 | GMTEYKLVVVGADGVGKSALTIQLIQNHFVDEYDP | Ras protein or |
| | TIEDSYRKQVVIDGETCLLDILDTAGQEEYSAMRD | fragment thereof |
| | QYMRTGEGFLCVFAINNTKSFEDIHHYREQIKRVK | |
| | DSEDVPMVLVGNKCDLPSRTVDTKQAQDLARSYGI | |
| | PFIETSAKTRQGVDDAFYTLVREIRKHKEKMSKDG | |
| | KKKKKKSKTKC | |
| 2 | SKTSNTIRVFLPNKQRTVVNVRNGMSLHDCLMKAL | Raf protein or |
| | KVRGLQPECCAVFRLLHEHKGKKARLDWNTDAASL | fragment thereof |
| | IGEELQVDFLDHVPLTTHNFARKTFLKLAFCDICQ | |
| | KFLLNGFRCQTCGYKFHEHCSTKVPTMCVDWSNIR | |
| | Q | |
| 3 | MEHIQGAWKTISNGFGFKDAVFDGSSCISPTIVQQ | Full-length c-RAF |
| | FGYQRRASDDGKLTDPSKTSNTIRVFLPNKQRTVV | protein [P04049.1] |
| | NVRNGMSLHDCLMKALKVRGLQPECCAVFRLLHEH | |
| | KGKKARLDWNTDAASLIGEELQVDFLDHVPLTTHN | |
| | FARKTFLKLAFCDICQKFLLNGFRCQTCGYKFHEH | |
| | CSTKVPTMCVDWSNIRQLLLFPNSTIGDSGVPALP | |
| | SLTMRRMRESVSRMPVSSQHRYSTPHAFTFNTSSP | |
| | SSEGSLSQRQRSTSTPNVHMVSTTLPVDSRMIEDA | |
| | IRSHSESASPSALSSSPNNLSPTGWSQPKTPVPAQ | |
| | RERAPVSGTQEKNKIRPRGQRDSSYYWEIEASEVM | |
| | LSTRIGSGSFGTVYKGKWHGDVAVKILKVVDPTPE | |
| | QFQAFRNEVAVLRKTRHVNILLFMGYMTKDNLAIV | |
| | TQWCEGSSLYKHLHVQETKFQMFQLIDIARQTAQG | |
| | MDYLHAKNIIHRDMKSNNIFLHEGLTVKIGDFGLA | |
| | TVKSRWSGSQQVEQPTGSVLWMAPEVIRMQDNNPF | |
| | SFQSDVYSYGIVLYELMTGELPYSHINNRDQIIFM | |
| | VGRGYASPDLSKLYKNCPKAMKRLVADCVKKVKEE | |
| | RPLFPQILSSIELLQHSLPKINRSASEPSLHRAAH | |
| | TEDINACTLTTSPRLPVF | |
| 4 | MAALSGGGGGGAEPGQALFNGDMEPEAGAGAGAAA | Full-length b-RAF |
| | SSAADPAIPEEVWNIKQMIKLTQEHIEALLDKFGG | protein |
| | EHNPPSIYLEAYEEYTSKLDALQQREQQLLESLGN | [AAA35609.2] |
| | GTDFSVSSSASMDTVTSSSSSSLSVLPSSLSVFQN | |
| | PTDVARSNPKSPQKPIVRVFLPNKQRTVVPARCGV | |
| | TVRDSLKKALMMRGLIPECCAVYRIQDGEKKPIGW | |
| | DTDISWLTGEELHVEVLENVPLTTHNFVRKTFFTL | |
| | AFCDFCRKLLFQGFRCQTCGYKFHQRCSTEVPLMC | |
| | VNYDQLDLLFVSKFFEHHPIPQEEASLAETALTSG | |
| | SSPSAPASDSIGPQILTSPSPSKSIPIPQPFRPAD | |
| | EDHRNQFGQRDRSSSAPNVHINTIEPVNIDDLIRD | |
| | QGFRGDGGSTTGLSATPPASLPGSLTNVKALQKSP | |
| | GPQRERKSSSSSEDRNRMKTLGRRDSSDDWEIPDG | |
| | QITVGQRIGSGSFGTVYKGKWHGDVAVKMLNVTAP | |
| | TPQQLQAFKNEVGVLRKTRHVNILLFMGYSTKPQL | |
| | AIVTQWCEGSSLYHHLHIIETKFEMIKLIDIARQT | |
| | AQGMDYLHAKSIIHRDLKSNNIFLHEDLTVKIGDF | |
| | GLATVKSRWSGSHQFEQLSGSILWMAPEVIRMQDK | |
| | NPYSFQSDVYAFGIVLYELMTGQLPYSNINNRDQI | |
| | IFMVGRGYLSPDLSKVRSNCPKAMKRLMAECLKKK | |
| | RDERPLFPQILASIELLARSLPKIHRSASEPSLNR | |
| | AGFQTEDFSLYACASPKTPIQAGGYGAFPVH | |
| 5 | MEPPRGPPANGAEPSRAVGTVKVYLPNKQRTVVTV | Full-length a-RAF |
| | RDGMSVYDSLDKALKVRGLNQDCCVVYRLIKGRKT | protein [P10398.2] |
| | VTAWDTAIAPLDGEELIVEVLEDVPLTMHNFVRKT | |
| | FFSLAFCDFCLKFLFHGFRCQTCGYKFHQHCSSKV | |
| | PTVCVDMSTNRQQFYHSVQDLSGGSRQHEAPSNRP | |
| | LNELLTPQGPSPRTQHCDPEHFPFPAPANAPLQRI | |
| | RSTSTPNVHMVSTTAPMDSNLIQLTGQSFSTDAAG | |
| | SRGGSDGTPRGSPSPASVSSGRKSPHSKSPAEQRE | |
| | RKSLADDKKKVKNLGYRDSGYYWEVPPSEVQLLKR | |
| | IGTGSFGTVFRGRWHGDVAVKVLKVSQPTAEQAQA | |
| | FKNEMQVLRKTRHVNILLFMGFMTRPGFAIITQWC | |
| | EGSSLYHHLHVADTRFDMVQLIDVARQTAQGMDYL | |
| | HAKNIIHRDLKSNNIFLHEGLTVKIGDFGLATVKT | |
| | RWSGAQPLEQPSGSVLWMAAEVIRMQDPNPYSFQS | |
| | DVYAYGVVLYELMTGSLPYSHIGCRDQIIFMVGRG | |
| | YLSPDLSKISSNCPKAMRRLLSDCLKFQREERPLF | |
| | PQILATIELLQRSLPKIERSASEPSLHRTQADELP | |
| | ACLLSAARLVP | |

We claim:

1-157. (canceled)

158. A method for detecting a conformational change in a protein complex, the method comprising:
a) contacting a Ras protein or a fragment thereof with a Raf protein or fragment thereof, wherein the Ras protein or a fragment thereof comprises a label; and
b) detecting a change in signal generated by the label using a surface-selective technique upon a conformational change in a structure of a protein complex formed upon binding of the Ras protein or fragment thereof to the Raf protein or fragment thereof.

159. The method of claim 158, wherein the Ras protein or fragment thereof comprises an amino acid sequence with at least 90% sequence identity to the sequence set forth in SEQ ID NO. 1.

160. The method of claim 158, wherein the Ras protein is a full-length KRAS protein comprising at least one mutation.

161. The method of claim 160, wherein the mutation is an oncogenic mutation.

162. The method of claim 158, wherein the Ras protein comprises a prenyl group.

163. The method of claim 162, wherein the prenyl group is a farnesyl group.

164. The method of claim 158, further comprising tethering the protein complex on a lipid bi-layer, comprising the steps of (a) mixing equimolar amounts of the first protein Ras or a fragment thereof and the second protein Raf or a fragment thereof, thereby forming said protein complex; and (b) incubating said mixture on a lipid bilayer thereby tethering said protein complex to the lipid bilayer.

165. The method of claim 158, further comprising tethering the protein complex on a lipid bilayer, comprising the steps of (i) tethering the Ras protein or fragment thereof to the lipid bilayer and (ii) adding the Raf protein or fragment thereof to the first protein on the bilayer, thereby tethering the protein complex on the bilayer.

166. The method of claim 158, further comprising comparing a detectable signal generated by the label upon formation of the protein complex tethered to a lipid bilayer to (i) the detectable signal generated by the first protein tethered to the lipid bilayer without the second protein and (ii) the detectable signal generated by the label attached to the second protein tethered to the lipid bilayer without the first protein.

167. The method of claim 158, further comprising measuring a detectable signal generated by the label in the protein complex in the presence of (i) GTP or an analog thereof and (ii) GDP or an analog thereof, wherein the protein complex is tethered to the lipid bilayer.

168. The method of claim 158, further comprising adding an agent that induces a conformational change in the structure of said protein complex.

169. The method of claim 158, further comprising adding an agent that inhibits formation of the protein complex.

170. The method of claim 169, further comprising comparing (i) a first detectable signal generated by the protein complex upon contacting with the agent, wherein the protein complex comprises a label at a first amino acid residue on the Raf protein or fragment thereof to (ii) a second detectable signal generated by the protein complex upon contacting with the agent, wherein the protein complex comprises a label at a second amino acid residue on the Raf protein or fragment thereof; wherein the first amino acid is located in a different region of the Raf protein or fragment thereof than the first amino acid; and wherein the first detectable signal is generated upon a conformation change in the protein complex at the first site and the second detectable signal is generated upon a conformational change in the protein complex at the second site.

171. The method of claim 158, wherein the Raf protein or fragment thereof comprises an amino acid sequence with at least 90% sequence identity to the sequence set forth in SEQ ID NO. 2.

172. The method of claim 158, wherein the Raf protein or fragment thereof comprises a Ras-binding domain and a cysteine-rich lipid binding domain.

173. The method of claim 158, wherein the Raf protein or fragment thereof consists of a Ras-binding domain and a cysteine-rich lipid binding domain.

174. The method of claim 158, wherein the Raf protein or fragment thereof does not comprise a kinase domain.

175. The method of claim 158, wherein the Raf protein or fragment thereof does not comprise a hinge region domain.

176. The method of claim 158, further comprising measuring a detectable signal generated by the label in the protein complex in the presence of (i) GTP or an analog thereof and (ii) GDP or an analog thereof, wherein the protein complex is tethered to a lipid bilayer.
